(19) United States
(12) Patent Application Publication
(10) Pub. No.: US 2009/0048528 A1
(43) Pub. Date: Feb. 19, 2009

(54) System and methods for detecting ischemia with a limited extracardiac lead set

(76) Inventors: Bruce Hopenfeld, Salt Lake City, UT (US); Michael Sasha John, Larchmont, NY (US); Tim Fischell, Kalamazoo, MI (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/889,752

(22) Filed: Aug. 16, 2007

(51) Int. Cl. A61B 5/0472

(52) U.S. Cl. 600/516

(57) ABSTRACT

Disclosed is a system for detecting pathophysiological cardiac conditions from a reduced number of extracardiac leads. A right side lead measures the electrical signal between the middle superior chest region over the heart and inferior right torso position. A left side lead measures the electrical signal between the left precordial chest region and an inferior left lateral or posterior torso position. The lead montage is preferably chosen so that, regardless of patient position (e.g. supine, upright), negative ST segments and/or T waves are used to detect right coronary or left circumflex ischemia. Also, in these positions, reduced slope of the final deflection in the QRS can be used to detect these types of ischemia. To detect transmural ischemia, the system examines changes in QRS slopes, ST segment, T wave and the difference between the J point and the PQ potentials. In addition, for transmural ischemia associated with the left anterior descending artery, a proxy for the propagation time across the front of the heart is examined by comparing QRS features of the right side lead with QRS features of the left side lead. Histogram profiles, trends, and statistical summaries, especially running averages, of all of the above mentioned features, corrected for heart rate, are maintained.

### FIELD OF USE

This invention is in the field of medical device systems that monitor a patient's cardiovascular condition.

### BACKGROUND OF THE INVENTION

Heart disease is the leading cause of death in the United States. A heart attack, also known as an acute myocardial infarction (AMI), typically results from a blood clot or “thrombus” that obstructs blood flow in one or more coronary arteries. AMI is a common and life-threatening complication of coronary artery disease. Coronary ischemia is caused by an insufficiency of oxygen to the heart muscle. Ischemia is typically provoked by physical activity or other causes of increased heart rate when one or more of the coronary arteries is narrowed by atherosclerosis. AMI, which is typically the result of a completely blocked coronary artery, is the most extreme form of ischemia. Patients will often (but not always) become aware of chest discomfort, known as “angina”, when the heart muscle is experiencing ischemia. Those with coronary atherosclerosis are at higher risk for AMI if the plaque becomes further obstructed by thrombus.

There are a number of portable monitors that attempt to detect AMI. Monitors that include wearable sensors (e.g. a medical-vest with electrodes) may be somewhat inconvenient for patients. Chronically implanted sensors provide the possibility for continuous monitoring without many of the inconveniences associated with wearable monitors. One type of implantable monitor includes an electrode chronically implanted within the heart. An intracardiac electrode may provide a strong signal at the cost of requiring intracardiac implantation. Another type of implantable monitor can rely upon subcutaneous electrodes, which are less invasive, but receive smaller amplitude signals compared to intracardiac electrodes.

Furthermore, subcutaneous electrodes require lead structures to connect them to the monitoring device. If the lead is also subcutaneous, it is generally desirable to keep it as short as possible. Shorter leads provide a more limited view of the torso's electrical field, which may in turn compromise the ability of a monitoring device to detect certain types of cardiac events. It would be desirable to have a subcutaneous electrode and lead system with relatively short leads that can diagnose a variety of cardiac conditions, including ischemia associated with significant occlusions of any of the three major coronary arteries, the left anterior descending artery, the left circumflex artery and the right coronary artery.

A number of different electrode configurations have been employed by existing surface electrode systems which are used for continuous patient monitoring. A five electrode system known as EASI can be used to derive data similar to that which would usually require a 12 lead electrocardiogram, by appropriate use of linear transformations. In this system, there are four unipolar electrodes and one ground electrode, which can be placed anywhere. Jahrsdoerfer M, Giuliano K, Stephens D, Clinical usefulness of the EASI 12-lead continuous electrocardiographic monitoring system. Crit Care Nurse. 2005 October; 25(5):28-30, 32-7.

There are various algorithms for detecting AMI by visual inspection of 12 lead ECGs (see, e.g. Use of the Electrocardiogram in Acute Myocardial Infarction, Zimetbaum P and Josephson, M, NEJM, 348:933-940 (2003)). Many such algorithms are based at least in part on ST segment shifts.

A body surface mapping approach for detecting both exercise induced and chronic ischemia based on activation sequence metrics rather than ST changes has been described by Igarashi et al (H Igarashi, M Yamaki, I Kubota, K Ikeda, M Matsui, K Tsuiki and S Yasui, Relation between localization of coronary artery disease and local abnormalities in ventricular activation during exercise tests Circulation, Vol 81, 461-469, 1990). This study involved constructing activation maps for the torso surface in normal and coronary artery disease (CAD) patients both before and after exercise. Local activation time for each of the 87 unipolar electrodes was defined as occurring at time of the maximum negative deflection of the corresponding electrode waveform. The global activation time for this electrode was defined as the difference between this local activation time and the QRS onset, which was determined from superimposed Frank X, Y and Z leads. The resulting activation maps of the CAD patients during exercise were compared with the exercise maps of normal patients during exercise. Further, the difference between CAD patients' exercise and resting activation maps was compared with the difference maps for normal patients.

Regarding subcutaneous systems, Song et al. (Journal of Electrocardiology Volume 37, Supplement 1, October 2004, Pages 174-179 The feasibility of ST-segment monitoring with a subcutaneous device) disclose a subcutaneous cardiac monitor/alarm device that is designed to detect ST-segment deviations present in acute ischemia. There are four unipolar leads at the corners of a 3×6 cm rectangle situated within the left precordial region. These four unipolar leads are used to derive bipolar montages across the rectangle diagonals.

Chronic subcutaneous or surface monitoring is confounded by axis shifts (e.g., a move from supine to upright) and low frequency ST segment drift. In U.S. Pat. No. 6,397,100 to Stadler and Shannon, ST segment values are low pass filtered to ensure that very rapid changes, which may be caused by axis shifts, are not considered to be ST shifts caused by ischemia. Two different low pass filters are applied, resulting in two different filtered signal. One filtered signal is representative of very slow ST baseline drift. The other filtered signal is representative of the true ST level excluding high frequency axis shift. ST segment deviation indicative of ischemia is equal to the difference between the filtered signals. In U.S. Pat. No. 6,128,526 to Stadler, et al., axis shifts are detected by establishing expected ranges for the amplitude of the R-waves in different leads, and declaring an axis shift if the measured R-wave amplitude consistently falls outside of the expected range. Also in the '526 patent, if an axis shift is detected, the expected ranges of “noise detection” parameter values are broadened. These parameters are used to determine whether a cardiac cycle is too noisy to use for ischemia detection.

Pueyo et al. (“High-Frequency Signature of the QRS Complex across Ischemia Quantified by QRS Slopes”, Computers in Cardiology 2005; 32:659-662) describe a method for examining the R and S wave slopes and amplitudes, associated with the standard 12 lead electrocardiogram leads, during balloon occlusions. Another study found that QRS amplitude changes during acute occlusion were overall more sensitive/specific than QRS slope and ST segment changes. Dori G, Denekamp Y, Fishman S, Rosenthal A, Frajewicki V, Lewis B S, Bitterman H “Non-invasive computerised detection of acute coronary occlusion.” Med Biol Eng Comput. 2004 May; 42(3):294-302.

The heart rate corrected QT interval has also been shown to be an indicator of early transmural ischemia. Kenigsberg D N, Khanal S, Kowalski M, Krishnan S C. “Prolongation of the QTc interval is seen uniformly during early transmural ischemia.” J Am Coll Cardiol. 2007 Mar. 27; 49(12):1299-305.

The Selvester QRS score estimates the size of a myocardial infarction based on QRS characteristics in various leads commonly used to procure a standard ECG. The score is a function of the duration of the Q and R waves and on the ratios of R-to-Q and R-to-S wave amplitude in all leads. (Wagner G S, Freye C J, Palmeri S T, Roark S F, Stack N C, Ideker R E, Harrell F E Jr, Selvester R H. Evaluation of a QRS scoring system for estimating myocardial infarct size. I. Specificity and observer agreement. Circulation. 1982 February; 65(2):342-7.) The Athens score for detecting ischemia is based on a comparison of the sum of the Q, R and S wave amplitudes (R-Q-S) before and during (or after) exercise. (Michaelides A P, Triposkiadis F K, Boudoulas H, Spanos A M, Papadopoulos P D, Kourouklis K V, Toutouzas P K. New coronary artery disease index based on exercise-induced QRS changes. Am Heart J. 1990 August; 120(2):292-302.)

U.S. Pat. No. 6,217,525 to Medema et al. describes a reduced lead set device (i.e. less than 12 leads) for detecting acute ischemia by separately analyzing features (e.g. ST elevation, T wave amplitude and QRS area) for each lead and/or analyzing a vector comprising concatenated heart beat information from a number of leads. Medema et al. describe both statistical and heuristic methods for detecting acute ischemia.

U.S. patent application publication number 20060253164 to Zhang et al. discloses a multi-lead system for detecting acute ischemia/infarction (among other event types) by calculating a “cardiac/QRS vector” and associating a change in the angle of this vector with ischemia/infarction. The “cardiac/QRS vector” is often referred to in the medical literature as the QRS axis, which represents the average direction of ventricular activation in the frontal plane (i.e. in a plane defined roughly by the front surface of a person's torso).

Because axis shifts can cause changes to wave segments of the ECG, such as the ST/T segment, this may induce spurious detections of ischemia in tests that examine the ST/T segment. Various schemes have been devised to distinguish between changes caused by axis shifts and true ischemic changes (e.g., ECG-based detection of body position changes in ischemia monitoring, Garcia, J.; Astrom, M.; Mendive, J.; Laguna, P.; Sornmo, L., IEEE Transactions on Biomedical Engineering, Volume 50, Issue 6, June 2003 Page(s): 677-685. F. Jager, R. Mark, G. Moody, and S. Divjak, “Analysis of transient ST segment changes during ambulatory monitoring using the Karhunen-Loeve transform,” in Comput. Cardiol., pp. 691-694, IEEE Comp. Soc., 1992. F. Jager, G. Moody, and R. Mark, “Detection of transient ST segment episodes during ambulatory ECG monitoring,” Comp. Biomed. Res., vol. 31, pp. 305-322, 1998). Some of these techniques rely on examining the abruptness of a an ECG signal change: axis shifts are thought to cause abrupt changes whereas ischemia is thought to cause more gradual and more persistent changes. Some of these techniques also rely on an extensive analysis of the QRS complex such as performing a principal component analysis on a reference set of beats to derive a set of basis QRS waveform shapes, and then decomposing beats to be tested into this set of basis shapes.

Smrdel A, Jager F (Automated detection of transient ST-segment episodes in 24 h electrocardiograms, Med Biol Eng Comput. 2004 May; 42(3):303-11) describe the concept of computing an ST segment reference (baseline). Ischemic episodes are based upon deviations of the ST segment from the baseline. A first estimate of the baseline is determined by performing long term averaging (loss pass filtering) of ST segment levels. If the Karhunen-Loeve coefficients of a number of consecutive beats indicate an axis shift has occurred, the ST segment reference changes rapidly so that it equals the new ST segment level, thereby adjusting to the new axis. Since the ST segment reference is equal to the ST segment value, there is no ST deviation. Thus, axis shifts are not characterized as ischemic.

Menown et al. describe a body surface mapping scheme, based on 64 unipolar electrodes, to detect AMI (“Body-surface map models for early diagnosis of acute myocardial infarction”, J. Electrocardiol. 1998; 31 Suppl:180-8.) Menown et al. implemented a multivariate test for AMI, where factor weights in the test were determined by regressions that compared normal patients to AMI patients. The test included factors pertaining to the QRS, ST and T waveform features. The factors in the test were also specific to electrode position (e.g. one factor was a QRS measure at a certain electrode while another factor was ST amplitude at a different electrode.)

Lehmann et al. (“Electrocadiographic algorithm for assignment of occluded vessel in acute myocardial infarction”, Int. Jnl. Cardiol., 2003, 89:79-85) describe an scheme that analyzes standard 12 lead ECG waveforms of patients with confirmed AMI to determine which artery is occluded. For example, ST elevation of greater than 0.1 mV in (standard 12 lead ECG) lead V2 results in a positive ischemia test for LAD occlusion whereas ST elevation less than 0.1 mV in this lead is classified as either LCX or RCA ischemia depending on the extent of ST changes in right side (augmented 12 lead) lead V5R. This scheme does not attempt to determine whether a patient has AMI. (Continuing with the above example, a patient with no ST changes in either lead V2 or lead V5R would be classified as having an LCX occlusion.)

Despite all of the foregoing work that has been done, there is still a need for an effective subcutaneous or surface based system for monitoring ischemia.

### SUMMARY OF THE INVENTION

An embodiment of the present invention comprises a system that includes of an implanted cardiac detection and/or diagnostic device and external equipment. The battery powered implantable cardiac diagnostic device contains electronic circuitry that can detect a cardiac event such as an acute myocardial infarction and warn the patient when the event, or a clinically relevant precursor, occurs. The cardiac diagnostic device can store the patient's electrogram for later readout and can send wireless signals to and receive wireless signals from the external equipment.

The cardiac diagnostic device receives electrical signals from subcutaneous or body surface leads. A right side lead measures the electrical signal between the middle superior chest region over the heart and inferior right torso position. A left side lead measures the electrical signal between the left precordial chest region and an inferior left lateral or posterior torso position. The lead montage is preferably chosen so that, regardless of patient position (e.g. supine, upright), negative ST segments and/or T waves are used to detect right coronary or left circumflex ischemia. Also, in these positions, reduced slope of the final deflection in the QRS can be used to detect these types of ischemia.

To detect transmural ischemia, the system examines changes in QRS slopes, ST segment, T wave and the difference between the J point and the PQ potentials. In addition, for transmural ischemia associated with the left anterior descending artery, a proxy for the propagation time across the front of the heart is examined by comparing QRS features of the right side lead with QRS features of the left side lead. Histogram profiles, trends, and statistical summaries, especially running averages, of all of the above mentioned features, corrected for heart rate, are maintained.

Axis shifts are determined by examining QRS shapes of the right and left leads and possibly information from other sensors such as level and acceleration detectors. If an axis shift causes a corresponding shift in a waveform feature value, the amount of the shift is subtracted from the waveform feature value to obtain a corrected waveform feature values. If there is significant variance in waveform feature values, especially QRS values, over a relatively small (e.g. 10) number of beats, the patient is assumed to be active and no attempt is made to correct for axis shifts.

Separate ischemia tests are applied for ischemia associated with the left anterior descending artery, left circumflex artery and right coronary artery. Each ischemia test is based upon the difference between the values of various features (e.g. final deflection slope) and the normative values. The normative values for a feature can depend upon posture, heart rate, and other parameters of a patient or appropriately matched population data, and the tests can be selected based upon these parameter values). The extent of the difference between actual and normal values is mapped to a likelihood of ischemia, which ranges between 0 and 1. The outcome of the ischemia test is the maximum likelihood of ischemia as determined over a number of sub-tests. A subtest may involve a single waveform feature value (e.g. final deflection slope) or a weighted combination of waveform feature values (e.g. final deflection slope and ST segment amplitude.) For example, if the final deflection slope alone corresponds to a likelihood of ischemia of 0.3 but the combined final deflection slope/ST subtest corresponds to a likelihood of ischemia of 0.5, then 0.5 is chosen as the overall likelihood of ischemia. In order to address multiple comparison effects, the number of subtests may be limited and the statistical probabilities may be adjusted according to the number of tests.

Methods are also disclosed for detecting subendocardial ischemia.

### BRIEF DESCRIPTION OF THE DRAWINGS

FIG. 1 illustrates a system for the detection of a cardiac event and for warning the patient that a medically relevant cardiac event is occurring;

FIG. 2 is a block diagram of an implanted cardiac diagnostic system according to the present invention.

FIG. 3a shows torso placement of right and left leads with respect to body surface potential distributions just before and just after the cardiac wave reaches the epicardial surface. FIG. 3b shows a hypothetical QRS complex recorded by the right lead.

FIG. 4 shows torso placement of right and left leads with respect to body surface potential distributions during the early and late QRS complex both before and during a balloon angioplasty of the left circumflex artery.

FIG. 5 shows torso placement of right and left leads with respect to body surface potential distributions during the ST segment in both healthy subjects and patients with posterior (left circumflex/LCX) transmural ischemia (STEMI).

FIG. 6a shows torso placement of right and left leads with respect to body surface potential distributions during the late QRS complex both before and during a balloon angioplasty of the left anterior descending (LAD) artery. FIG. 6b shows torso placement of right and left leads with respect to body surface potential distributions during the ST segment in both healthy subjects and patients with LAD STEMI.

FIG. 7a shows torso placement of right and left leads with respect to body surface potential distributions during the mid QRS complex both before and during a balloon angioplasty of the right coronary artery (RCA). FIG. 7b shows torso placement of right and left leads with respect to body surface potential distributions during the ST segment in both healthy subjects and patients with RCA/inferior STEMI.

FIG. 8 shows hypothetical waveforms recorded by the left lead in normal conditions and in conditions of a posterior/left circumflex STEMI.

FIG. 9 is a table that shows the types of changes expected in various final deflection form features in the case of LAD, LCX and RCA STEMI, respectively.

FIG. 10 shows a hypothetical plot of the running average of a waveform feature (e.g. final deflection slope) as a function of time.

FIG. 11 is a flow chart of the present invention's STEMI detection scheme implemented by the architecture shown in FIG. 2.

FIG. 12 is a flow chart of the anterior propagation time calculation implemented as part of the steps of the flowchart of FIG. 11.

FIG. 13 shows detection of long term, medium term and short term trends/changes in the running average of a waveform feature.

FIG. 14 shows an electrocardiogram waveform marked to show the definitions of varioufinal deflectionform features.

FIG. 15 shows an example of a sigmoidal function that relates the value of a waveform feature to the likelihood of ischemia.

FIG. 16 is a table that shows an example the components of an ischemia score that is based on the values of various waveform features.

### DETAILED DESCRIPTION OF THE INVENTION

“Lead” means at least two sensors that are configured to detect the electrical potential between two points.

“Primary deflection” means that portion of the QRS complex characterized by the largest amplitude peak to peak potential change. For example, in the context of a normal QRS complex recorded by precordial lead V3, the primary deflection is that part of the QRS that connects the peak of the R wave to the peak of the final deflection.

“Initial deflection” means that portion of the QRS complex before the primary deflection.

“Final deflection” means that portion of the QRS complex after the primary deflection.”

An “ischemia test” applied to a waveform feature value is a one or more mathematical operations performed on the waveform feature value and test parameter value(s). For example, if a waveform feature value is ST shift (x), an “ischemia test” is (x>0.1 mV), which is true when the ST shift is greater than 0.1 mV and otherwise false. Continuing with this example, a different ischemia test is (x>0.2 mV), i.e. the two tests are different even though they involve the same mathematical operation because a test parameter (0.1 mV vs. 0.2 mV) varies across the tests. An ischemia test (“composite ischemia test”) may be composed of a number of ischemia tests, in which case the composite ischemia test is preferably positive when any of the ischemia tests are positive, although this definition may also require that at least a selected number of the ischemia tests be positive.

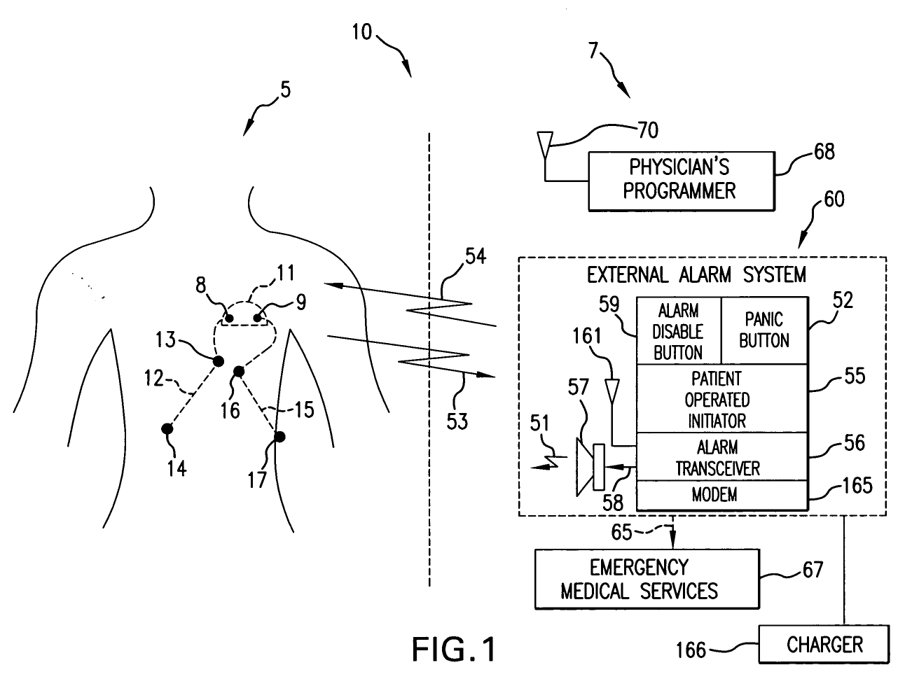

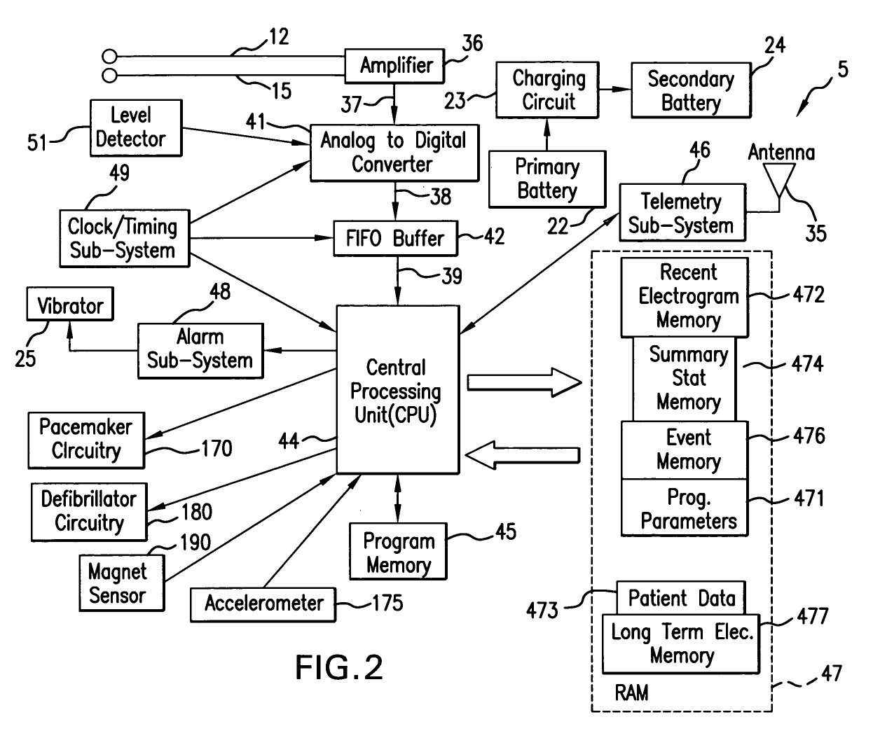

FIG. 1 illustrates one embodiment of a system 10 comprising an implanted cardiac diagnostic device 5 and external equipment 7. The battery powered cardiac diagnostic device 5 contains electronic circuitry that can detect a cardiac event such as an acute myocardial infarction or arrhythmia and warn the patient when the event, or a clinically relevant precursor, occurs. The cardiac diagnostic device 5 can store the patient's electrogram for later readout and can send wireless signals 53 to and receive wireless signals 54 from the external equipment 7. The functioning of the cardiac diagnostic device 5 will be explained in greater detail with the assistance of FIG. 2.

The cardiac diagnostic device 5 receives electrical signals from subcutaneous or body surface leads 12 and 15. Right side lead 12 comprises electrodes 13 and 14 with polarity hereafter defined as the difference potential measured between electrode 13 and electrode 14. Left side lead 15 comprises electrodes 16 and 17 with polarity hereafter defined as the potential at electrode 16 minus the potential at electrode 17. The right side lead 12 measures the electrical signal between the middle superior chest region over the heart and inferior right torso position. The left side lead 15 measures the electrical signal between the left precordial chest region and an inferior left lateral or posterior torso position. Electrode placement will be further described below. The cardiac diagnostic device 5 is housed in a metal case 11 that can serve as another electrode. In particular, the lead 15 could effectively be temporally multiplexed so that it alternately measures the potential across electrodes 16 and 17 and the potential across the metal case 11 and the electrode 16 (or 17).

FIG. 1 also shows the external equipment 7 that consists of a physician's programmer 68 having an antenna 70, an external alarm system 60 including a charger 166. The external equipment 7 provides means to interact with the cardiac diagnostic device 5. These interactions include programming the cardiac diagnostic device 5, retrieving data collected by the cardiac diagnostic device 5 and handling alarms generated by the cardiac diagnostic device 5. The operation of these components is further described in U.S. patent application publication number 2004/0215092.

FIG. 2 is a block diagram of the cardiac diagnostic device 5 with primary battery 22 and a secondary battery 24. The secondary battery 24 is typically a rechargeable battery of smaller capacity but higher current or voltage output than the primary battery 22 and is used for short term high output components of the cardiac diagnostic device 5 like the RF chipset in the telemetry sub-system 46 or the vibrator 25 attached to the alarm sub-system 48. According to a dual battery configuration, the primary battery 22 will charge the secondary battery 24 through the charging circuit 23. The primary battery 22 is typically a larger capacity battery than the secondary battery 24. The primary battery also typically has a lower self discharge rate as a percentage of its capacity than the secondary battery 24. It is also envisioned that the secondary battery could be charged from an external induction coil by the patient or by the doctor during a periodic check-up.

The pairs of wires corresponding to leads 12 and 15 respectively connect to the amplifier 36, which is a multi-channel or differential amplifier. The amplified electrogram signals 37 from the amplifier 36 are then converted to digital signals 38 by the analog-to-digital converter 41, which preferably samples at a rate of at least 500 Hz. The temporal resolution of the sampling is relevant with regard to the sampling of the high frequency components of a heartbeat's activation (QRS) complex, as will be further described below. The digital electrogram signals 38 are buffered in the First-In-First-Out (FIFO) memory 42. Processor means shown in FIG. 2 as the central processing unit (CPU) 44 coupled to memory means shown in FIG. 2 as the Random Access Memory (RAM) 47 can process the digital electrogram data 38 stored the FIFO 42 according to the programming instructions stored in the program memory 45. This programming (i.e. software) enables the cardiac diagnostic device 5 to detect the occurrence of a cardiac event such as an acute myocardial infarction.

A level detector 51 is coupled to the analog to digital converter 41. The level detector 51 detects whether a patient's torso is upright or supine and also, if the torso is supine, the extent of its rotation with respect to the earth (e.g. patient is lying flat on his/her back, lying on his/her right side or left side.) Many MEMS based inclinometers/level detectors exist.

Additional sensors may communicate with the device 5 wirelessly through the telemetry Sub-system. The data from these leads may correspond to digitized electrogram signals (that have been processed by a remote subcutaneous device).

The operation of most of the components in FIG. 2 is further described in U.S. patent application publication number 2004/0215092.

In a preferred embodiment of the present invention the RAM 47 includes specific memory locations for 4 sets of electrogram segment storage. These are the recent electrogram storage 472 that would store the last 2 to 10 minutes of recently recorded electrogram segments so that the electrogram data occurring just before the onset of a cardiac event can be reviewed at a later time by the patient's physician using the physician's programmer 68 of FIG. 1. For example, the recent electrogram storage 472 might contain eight 10-second long electrogram segments that were captured every 30 seconds over the last 4 minutes.

A summary statistics memory 474 would provide storage for summary information, such as running averages, of various cardiac waveform feature values. A long term electrogram memory 477 would provide storage for electrograms collected over a relatively long period of time. In the preferred embodiment, every ninth electrogram segment that is acquired is stored in a circular buffer, so that the oldest electrogram segments are overwritten by the newest one.

The telemetry sub-system 46 with antenna 35 provides the cardiac diagnostic device 5 the means for two-way wireless communication to and from the external equipment 7 of FIG. 1. Existing radiofrequency transceiver chip sets such as the Ash transceiver hybrids produced by RF Microdevices, Inc. can readily provide such two-way wireless communication over a range of up to 10 meters from the patient. It is also envisioned that short range telemetry such as that typically used in pacemakers and defibrillators could also be applied to the cardiac diagnostic device 5. It is also envisioned that standard wireless protocols such as Bluetooth and 802.11a or 802.11b might be used to allow communication with a wider group of peripheral devices.

A signaling device which may be in the form of a telemetry mechanism 46 is in communication with processor unit 44 for sending a signal to an indicator device which may be any of a number of commercially available audio or visual indicators providing a display or audio indication of the signal being sent when a pathological heart condition is detected. The telemetry mechanism may include a wireless transmitter or cell phone well known in the art.

Additionally the signaling device may be an alarm system 60 which may be external the patient.

Electrode Positions

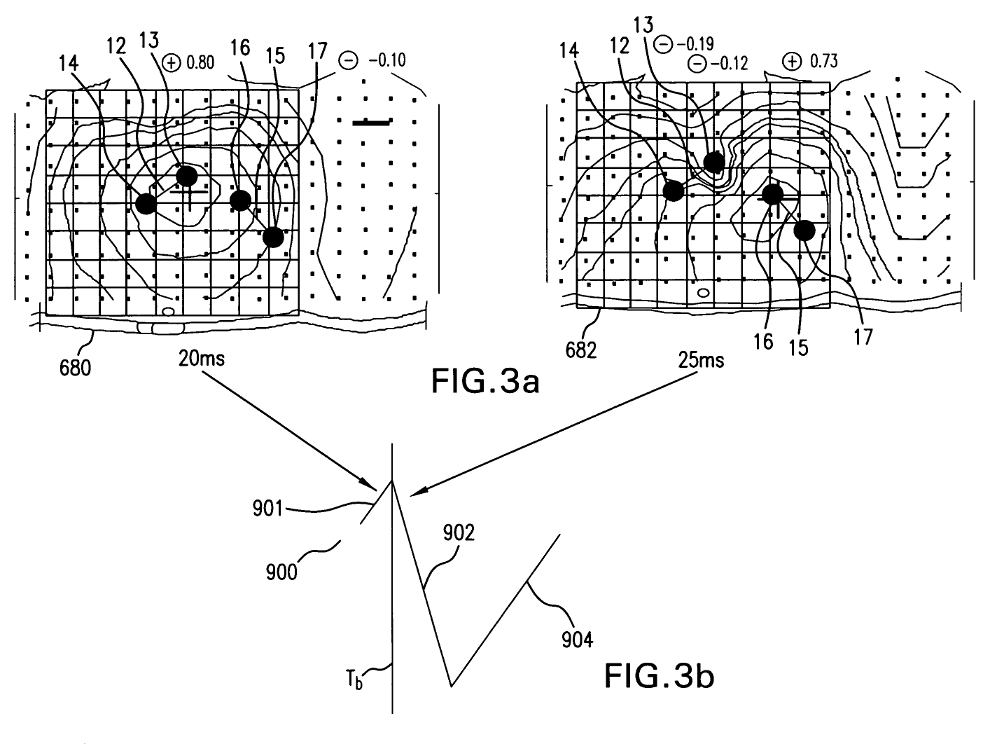

Orientations of the leads and the corresponding positions of electrodes 13, 14, 16 and 17 (FIG. 1) will be described with reference to FIGS. 3-7, which show body surface maps from various reported experiments. A grid has been overlaid on the torso drawings to provide for a common coordinate system amongst the different torso drawings. Optimal electrode positioning is preferably patient dependent; if a patient has had body surface measurements taken during balloon angioplasty, electrode positions may be set in part based on this data, according to the principles outlined below. Even if no such intervention has been done, it is nonetheless desirable to obtain body surface measurements from a patient to determine improved electrode positions in the manner described below.

FIG. 3a shows body surface maps 680 and 682 from Miller et al. (Total Body Surface Potential Mapping During Exercise: QRS-T-wave Changes in Normal Young Adults, Circulation; 62(3):632-645, 1980) of a healthy resting individual during the early portion of the QRS complex, just before (680) and after (682) the cardiac wave has first reached the epicardial surface (‘breakthrough’). The torso is ‘cut’ along the right side and then unrolled, so that the left portion of the drawing represents the anterior torso and the right portion of the drawing represents the posterior torso (all of the body surface maps shown in FIGS. 3-7 show the torso in this manner). The positions of the neck and shoulders are indicated at the tops of the body surface maps. The values of the maximum and minimum potentials are shown (in mV) at the tops of the maps In the right side panel, there are two minima with associated potential values of −0.19 mV (minimum near the throat) and −0.12 mV (minimum partly obscured by electrode 13), respectively.

Lead 12 and associated electrodes 13 and 14 would be expected to record a QRS final deflection waveform similar to waveform 900 shown in FIG. 3b. During early QRS, before 20 ms (map 680) and a few ms thereafter, there will be a positive deflection (defined herein as the initial deflectiondeflection) 901. After breakthrough, there will be a strong negative deflection (defined herein as the primary deflection) registered by lead 12, as shown in map 682 and indicated by the primary deflection 902 in FIG. 3b. The breakthrough-time (T_{b}) may be defined as the peak of the initial deflection 901. It is desirable for electrode 13 to be positioned in a region where a patient's breakthrough is manifest on the torso surface (i.e. at a location on the anterior chest that is among the first to register negative potentials.)

The waveform recorded by lead 15 will show an initial small upstroke (initial deflection) during early QRS as indicated by map 682. Breakthrough will not cause any significant deflection in this lead, as may be seen by comparing map 682 with map 680. The 20 and 25 msec time markers indicate the time from the onset of the QRS complex.

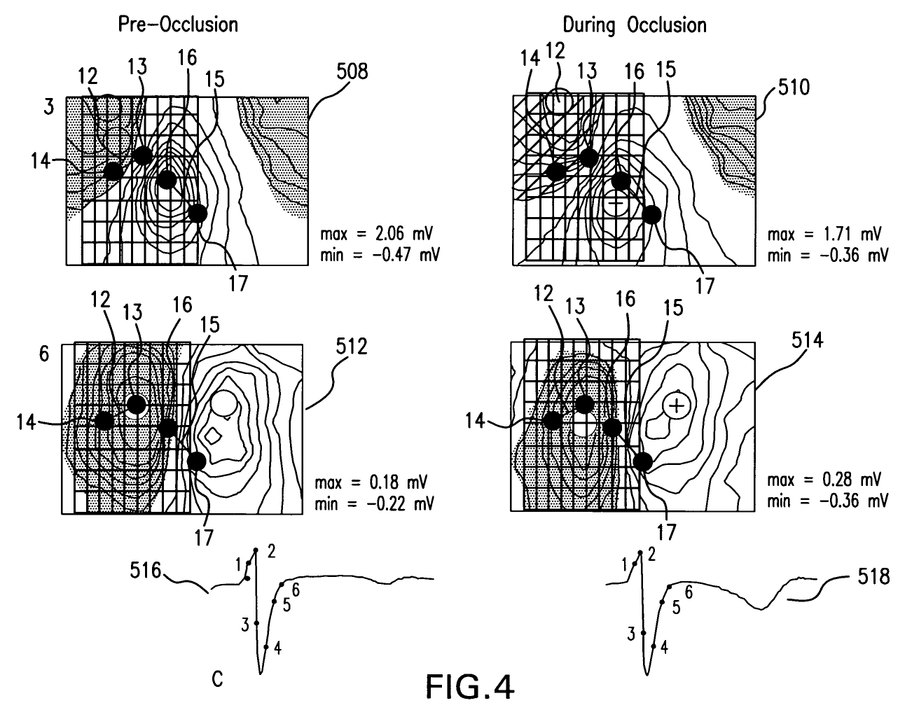

FIG. 4 shows body surface maps during the QRS complex in a patient before and during left circumflex (LCX) balloon angioplasty, which tends to mimic the conditions of an acute, transmural ischemic event. These maps are from Spekhorst et al. (Body surface mapping during percutaneous transluminal coronary angioplasty. QRS changes indicating regional myocardial conduction delay, Circulation. 1990 March; 81(3):840-9.) The contour intervals vary across the maps 508, 510, 512 and 514. Waveforms 516 and 518 provide a temporal reference that shows when the body surface maps 508, 510, 512 and 514 were recorded within the QRS complex. Waveforms 516 and 518 show recordings from the anterior chest region before and during occlusion respectively. In the maps, the darker shadings represent negative polarities and the lighter colors are positive polarities, with the minimum and maximum indicated with “−” and “+” symbols, respectively. Maps 508 and 510 were recorded at a time (mid-QRS) indicated by the numeral three in waveforms 516 and 518 while maps 512 and 514 were recorded at a time (late QRS) indicated by the numeral 6 in waveforms 516 and 518.

A comparison of the mid-QRS maps 508 and 510 suggests that the potential drop across lead 15 is relatively smaller during an LCX occlusion. However, theory suggests that it is also possible for this lead to register a larger mid-QRS potential drop during LCX occlusion. Thus, both positive and negative mid-QRS deviations, which show more than a specified difference when compared to a patient's self norm data, across lead 15 may be indicators of LCX ischemia (and possibly other pathophysiological conditions). At this mid-QRS time, the torso projection of the cardiac wavefront has not reached electrode 16. When the torso projection of the cardiac wavefront crosses electrode 16, there will be a large negative deflection across lead 15. At this stage of the QRS complex, the cardiac wavefront essentially moves from right to left across the anterior portion of the heart. This results in a corresponding spatially smoothed “wavefront” that moves from right to left across the anterior torso. On the body surface maps in the figure, this projected “wavefront” is indicated by the area of steep contours between the potential maximum and minimum.

In the late QRS (maps 512 and 514), the cardiac wavefront projection has reached and passed electrode 16. As the cardiac wave propagates through the posterior portion of the heart, the potential registered by electrode 17 becomes less positive, so that the waveform recorded by lead 15 shows an upstroke (final deflection) during this time. In conditions of a transmural posterior (LCX) ischemia, the upstroke will be slower (since the cardiac wave travels more slowly through the posterior ischemic tissue) compared to the case when the tissue is healthy. In LCX ischemia the resulting final deflection slope is relatively smaller, and the potential across lead 15 will usually be relatively more negative at any given time in the late QRS. This is evident by comparing the total (maximum−minimum) potential drop of 0.40 mV (0.18 mV−(−0.22 mV)) in the pre-occlusion map 512 with the drop of 0.64 mV in the occlusion map 514. In this latter case it should be noted that the lead 15 measures a portion of the potential drop rather than the total potential drop.

Lead 15 should be aligned as closely as possible with the late QRS maximum and minimum, in order to detect LCX ischemia based on QRS features. In one embodiment, in the case where there are only a small number (e.g. 2) of leads sensing cardiac activity of a patient, lead 15 is preferably not exactly aligned with this maximum and minimum since this position would compromise the recording of other important features of the projected waveform, as will be further described below. When more that 2 leads are used, one or more leads may be used to detect a torso pattern which is likely to indicate a particular type of ischemia (e.g. LCX). When this pattern is detected in a patient, the data from the an additional lead or (leads) is then sensed and analyzed in order to increase the specificity of the detections, by ensuring that the additional spatiotemporal topographies are present prior to marking the activity as ischemic. The selective use of additional leads decreases the computational and sensing requirements of the system during normal use, and increases operation when the detection leads determine abnormal activity may be occurring.

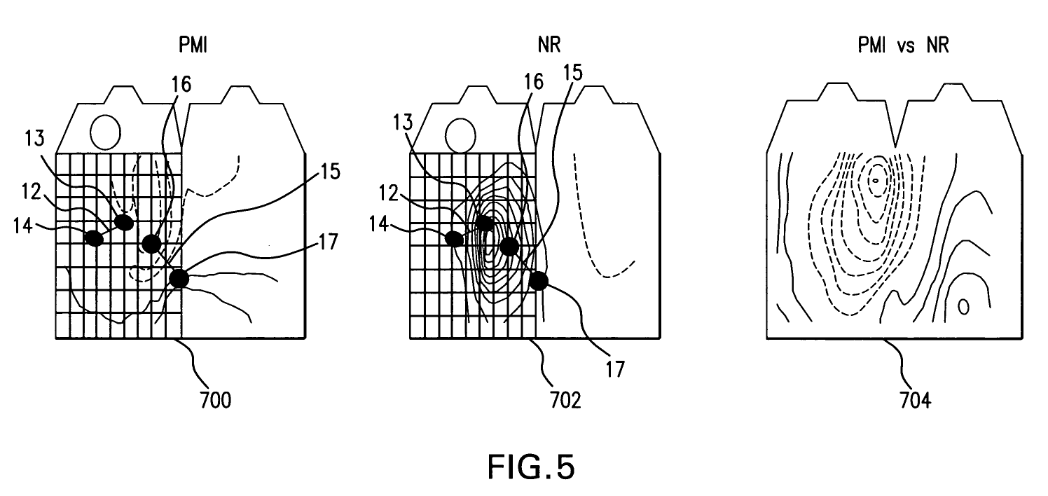

FIG. 5 shows averaged body surface maps of the ST segment potentials of persons diagnosed with posterior ST elevation ischemia (“PMI” as seen in map 700) and healthy subjects (“NR” as shown in map 702). (Kornreich et al., Body surface potential mapping of ST segment changes in acute myocardial infarction. Implications for ECG enrollment criteria for thrombolytic therapy. Kornreich F, Montague T J, Rautaharju P M. Circulation. 1993 March; 87(3):773-82.) Map 704 shows the difference between posterior ST elevation ischemia (“STEMI”) patients and healthy subjects (effectively map 700-map 702). In maps 700 and 702, contour intervals are 20 μV while in map 704, the contour intervals are in units of a statistical measure based on standard deviation. Positive potentials are indicated by continuous lines while negative potentials are indicated by dotted lines. The lead 15 will record positive ST segment potentials in the case of normal subjects and negative ST segment potentials in the case of posterior/LCX STEMI subjects. The lead 12 will record a similar but smaller ST transition from positive to negative during a transition from normal to posterior ischemia.

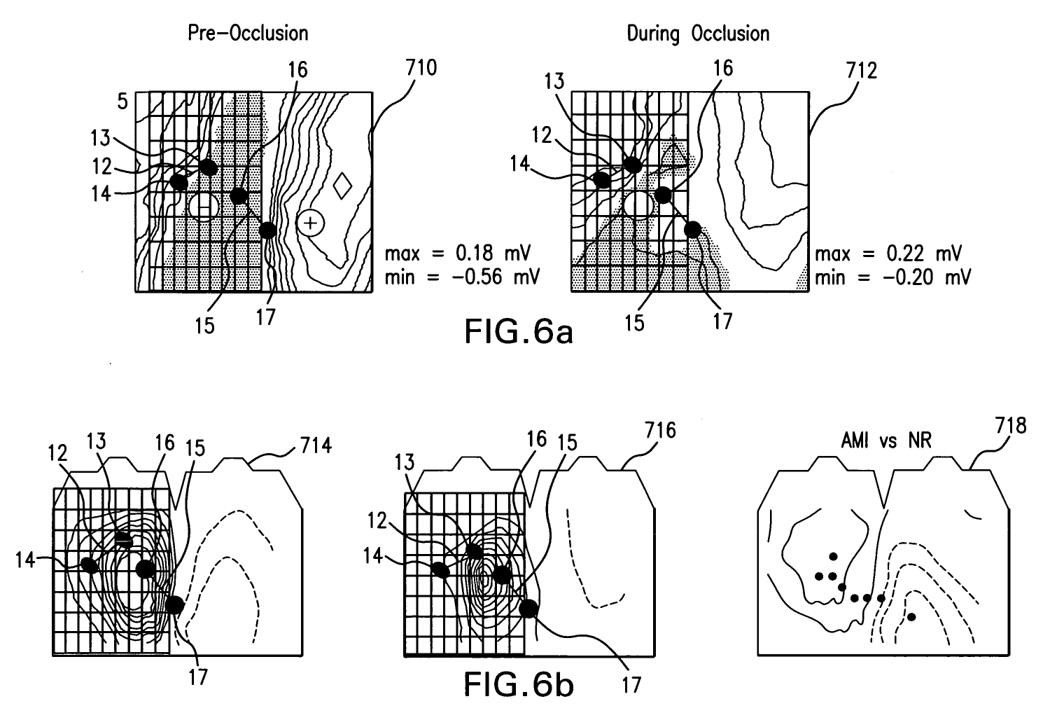

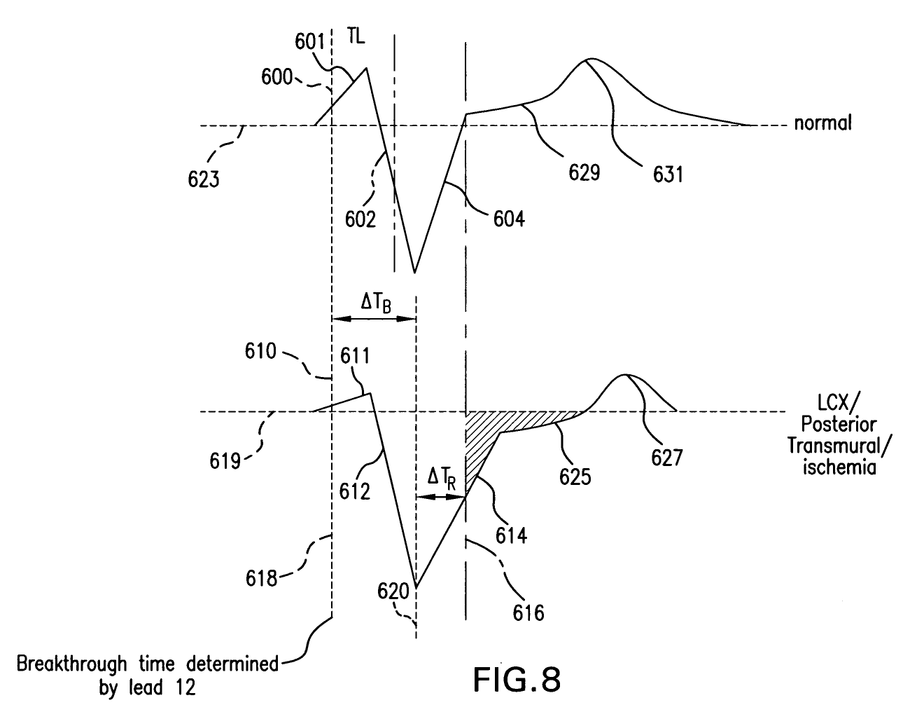

FIG. 6a shows mid/late QRS potentials from Spekhorst et al. (1990) before (map 710) and during (map 712) a balloon occlusion of the left anterior descending artery (LAD). Mid/late in the QRS (e.g. 50 ms after QRS onset), in the normal case (no STEMI), the lead 15 has registered a large negative deflection (primary deflection) due to normal propagation of the cardiac wave (map 710). However in the case of an anterior STEMI, measured with respect to the same fiducial time marker (60 ms after QRS onset in the above example), the lead 15 has not recorded a large negative deflection but rather a positive one (map 712). The time (Tₗ) of the negative deflection (primary deflection) of lead 15, as indicated by the dash-dot-dot line in FIG. 8, may thus be used a proxy for the timing of propagation across the anterior heart. The timing of breakthrough (T_{b}), described with reference to FIG. 3, may be used as a reference time for the onset of propagation across the anterior heart. Propagation time defined as Tₗ−T_{b}, both recorded from lead 15, can be a measure for assessing anterior STEMI, where as this interval increases above a selected value an occurrence of STEMI may be detected. This propagation time will normally be in the range of 20 ms-25 ms, depending on the person and the position of electrode 16. The extent of propagation slowing caused by ischemia varies according to many factors, but an estimate of 25%-40% slowing is reasonable. Thus, a large area of transmural anterior ischemia may cause a 5 ms-10 ms delay in the propagation time Tₗ−T_{b}, relative to the non-ischemic propagation time

Maps 714 and 716 in FIG. 6b show ST segment potentials from Kornreich et al. in the case of anterior STEMI patients and healthy subjects, respectively. The map of the normal controls NR 716 is identical to map 702 shown in FIG. 5 (i.e. these maps were derived from the same patients). Map 718 shows the different between the anterior STEMI and healthy subjects. Leads 12 and 15 will register ST elevation in both the normal and STEMI cases although the elevation will be greater in the case of STEMI.

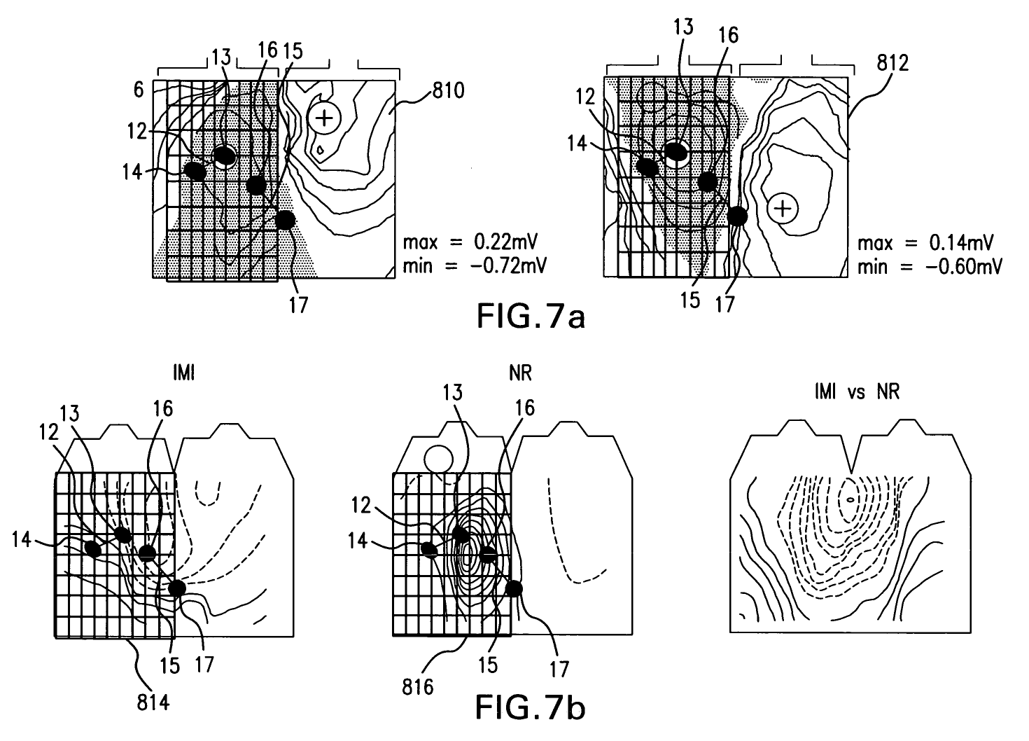

FIG. 7a, based on maps from Spekhorst et al., shows late QRS potentials before (map 810) and during (map 812) an RCA balloon occlusion. These maps suggest that there may be some RCA occlusion induced difference in the QRS waveforms recorded by leads 12 and 15. FIG. 7b, based on maps from Kornreich et al., shows group average ST segment maps for RCA STEMI patients (map 814) and normal patients (map 816). Both leads 12 and 15 will register negative ST potentials during an RCA STEMI compared to positive potentials during the normal case.

FIG. 8 shows hypothetical waveforms recorded by lead 15 in the normal case (waveform 600) and in the case of a posterior/LCX STEMI (waveform 610). The dotted lines 623 and 619 depict the reference potential. The initial deflection 611 is characterized by a reduced slope compared to the initial deflection 601 in normals (waveform 600), at least according to the data of Spekhorst et al., while the primary deflection 612 is smaller (i.e. the extent of the negative deflection) than the primary deflection 602. The slope of the following final deflection 614 is smaller than the slope of final deflection 604, and is below the reference voltage after the time the normal waveform 600 has turned positive (due to anterior repolarization), as indicated by the dash-dot line. The ischemic ST segment 625 is relatively negative with a reduced slope compared to the healthy ST segment 629, and the ischemic T wave 627 has a reduced amplitude compared to the healthy T wave 631. Theory suggests that T wave amplitude will generally be reduced according to the severity of the ischemia, with severe ischemia possibly causing flat or inverted T waves.

Ischemia metrics in addition to the ones mentioned above (e.g. final deflection slope) may be generated with respect to timing information. The normal time that the ST segment turns positive (dash dot line 616) may be defined with respect to breakthrough time (ΔT_{B}) and/or the nadir of the R wave 620 (ΔT_{R}). A metric for ischemia could then be defined as the summed or averaged value of a portion of waveform 610 starting at the dash-dot line 616 and ending at the end of the ST segment, as indicated by the area marked with diagonal lines. Instead of the end of the ST segment, the peak T wave, or end T wave could be used. If anterior ischemia is also present, the final deflection 614 will be even further delayed (i.e. will occur later because the primary deflection 612 was delayed), so that integration/averaging of the waveform after the time point defined by ΔT_{B} will be even more negative than in the case of posterior ischemia alone. Another useful metric is the time between some fiducial time ΔT_{B} and the time required for the waveform to reach a value such as crossing the zero line (e.g. reference potential) and turning positive.

In an AC coupled system, voltages are measured with respect to a chosen reference potential 619. If this reference is chosen as the TQ segment, as is typical, and ischemia is present, then there is current flow during the TQ segment even though the voltage level is assigned a value of zero. The current flow during the TQ segment is in a direction opposite to the current flow during the ST segment.

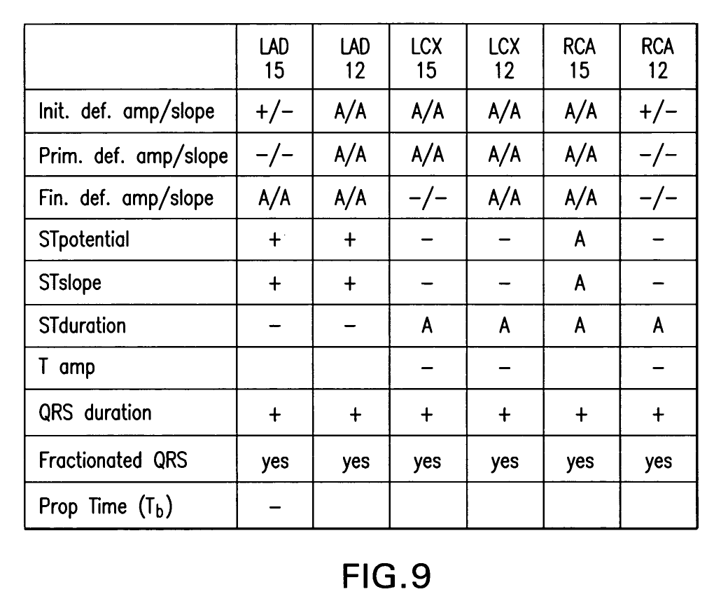

FIG. 9 is a table that shows expected changes to various heart signal features in the case of LAD, LCX and RCA ischemia. The term ‘amp’ as applied to a wave refers to the difference between the maximum and minimum potentials associated with the particular wave, not the absolute maximum or minimum potential across the QRS complex. The ‘+’ and ‘−’ symbols mean that the heart signal feature would be expected to increase and decrease, respectively, relative to a baseline or normal condition (e.g. a self norm or a population norm). For example, if a person's normal primary deflection amplitude is 2 mV, and a value of 1.4 mV is indicative of LAD ischemia, then there will be a minus sign in the table for primary deflection amplitude for LAD ischemia.) The ‘A’ symbol means that expected changes are indeterminate; in the case of an ‘A’ designation, any significant change (e.g. +/−2 standard deviations from the mean normal value), whether positive or negative, is factored into the analysis.

The amplitudes of the final deflection initial, primary and final deflections, the expected propagation time, the slope of the ST segment, and the shape and magnitude of the T wave are all heart rate dependent. For example, depending on the position of electrode 16, there may be an increase in initial deflection amplitude and slope with heart rate in healthy subjects, as suggested by data from Miller et al. (Circulation; 632-645, 1980). There would be generally be a greater increase in initial deflection amplitude/slope when the potential is measured across the metal case 11, which is superior to the electrode 16, and the electrode 17 (see FIG. 1). The above mentioned changes are preferably evaluated by adjusting relevant parameter values and deviations according to heart rate.

The adjustment of parameter values and deviations may be performed by selecting appropriate values according to assessment of recent cardiac activity by assessing different bins of a heart rate histogram (as is disclosed in U.S. patent application publication number 20070093720 to Fischell et al.) Each separate parameter value of the table may be adjusted according to a linear or non-linear function which varies with heart rate. This type of patient-specific adjustment may be efficiently derived by taking measurements at several selected heart rates for a particular patient, and fitting polynomials to the resulting data points. Body surface mapping of a patient before, during and after upright and supine exercise may help to establish expected waveform values for that patient across a range of heart rates and postures.

### Axis Shifts—Beat Types

Since the heart can move relative to the torso, the waveforms recorded by leads 15 and 12 will change according to a patient's posture. Most pertinently, in an upright posture, the QRS potentials are shifted upward in the torso compared to a supine posterior. While ST potential patterns are similar between upright and supine, the magnitudes are larger in the supine position (Sutherland et al., Am J Cardiol 1983; 52:595-600). The body surface potentials don't change drastically with normal breathing but do change significantly between maximal and minimal lung capacity.

Furthermore, in the patient's new position (e.g. lying down instead of standing), it is desirable that the ischemia tests that are applied, which depend on the expected values of various posture/patient state dependent heart signal features (e.g. ST segment potential), are appropriate and do no result in false negatives.

To determine the appropriate parameters to apply to a given beat, which is associated with a particular patient posture (or more generally patient state), the patient posture/patient state is estimated from the QRS waveforms recorded by the leads 15 and 12 and possibly other patient state parameters, if available, such as tilt information from the level/tilt sensor 51 in FIG. 2. The determined patient posture/patient state will be referred to as a “Beat Type.” To take the simplest example, there may be two Beat Types, one for a supine posture and one for an upright posture. The QRS waveforms associated with these postures may be determined by recording the waveforms seen by these electrodes while the patient is supine or upright respectively, preferably at different heart rates, to generate template QRS waveforms for these two (preferably heart rate dependent) Beat Types. Both normal and abnormal criteria can be derived for various beat types (e.g. during a calibration testing procedure) and can be used when the patient has assumed different postures (e.g., sitting, standing, lying on the left or right side, or on stomach or on back). Further, the particular tests which are applied to the cardiac features may change as a function of beat type. In this manner, the features which are evaluated, the tests which are applied, and the criteria of these tests can be altered according to beat type in order to increase the sensitivity and specificity of the events which are detected.

An electronic tilt/level detector 51 can be used to determine if the patient is standing/sitting or lying down. The detector 51 can be integrated with (or also function as) an accelerometer in order to detect transitions, and the acceleration of transitions, from laying down to sitting or standing. The detector may contain a number of technologies (e.g. MEMS) which permit assessment of posture for example by detecting the direction of gravitational pull. Since the information derived from the detector 51 can depend upon the orientation of the implanted device (or implanted detector if this is self contained), which may shift over time, an ‘orientation and movement calibration protocol’ can be implemented wherein the patient is asked to lie down, sit, and stand up, etc. in order to calibrate the settings of these components. In order to improve the reliability of the readings, the implantable device may be programmed to issue “status” signals which tell the patient that the device is measuring the patient's activity correctly. For example, 3 quick high tones can be emitted from the device when the patient first stands up, while 3 quick low tones are emitted when the patient lies down. The patient can use the external patient programmer to increase or decrease sensitivity or to indicate whether the status signals are correct or incorrect and the program may be configured to use this patient feedback to increase its performance. This type of calibration can be scheduled to occur once a day or otherwise. Lastly in addition to the detector 51, additional detectors can be located in various locations (e.g. near the distal end of an intracardiac electrode) in order to measure for example, a patients posture, the orientation of the heart within the patient's chest cavity, and the orientation of the heart with respect to gravity. Further, a remote detector can be implanted in a patient's leg in order to differentiate sitting from standing, as this may be important to detect some cardiac states in a small subgroup of the patient population. In addition to changes in posture, the cumulative time which has transpired since a transition in posture may also be used to adjust the tests applied to the features of cardiac data.

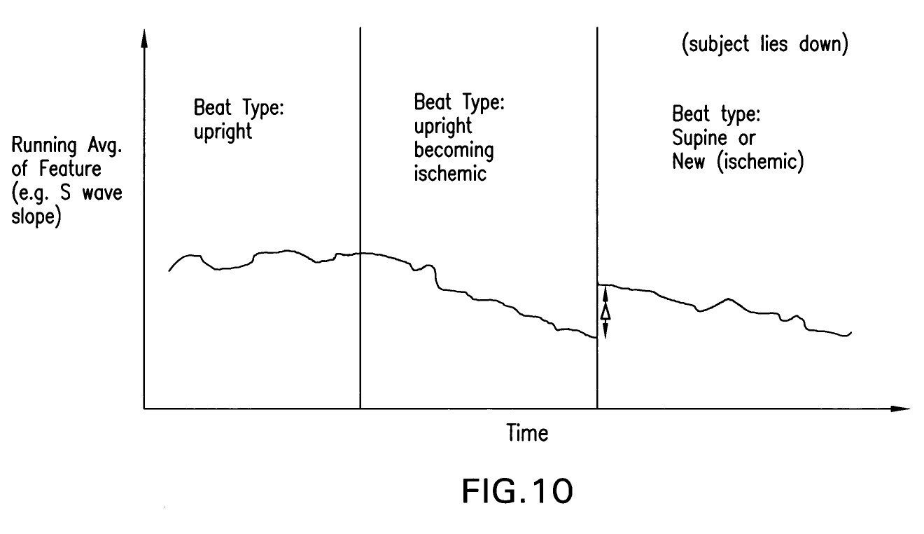

FIG. 10 shows a hypothetical plot of the running average of a waveform feature (e.g. final deflection slope) as a function of time. Initially, the patient is assumed to be upright (left panel) and the Beat Type is correspondingly designated as upright. The patient, while upright, becomes ischemic, and the final deflection slope decreases (middle panel), but not enough to trigger an alarm. The patient then lies down (right panel), which causes an abrupt change in the final deflection slope. The running average of the final deflection slope is reset, so that there is a sharp boundary between the upright and new Beat Types. If it is possible to determine whether the patient is lying down (e.g. by means of level sensor 51 in FIG. 2), then the Beat Type is supine. Otherwise, the system may not be able to determine that the patient is supine by analyzing QRS features, since ischemia has caused a substantial change in the QRS. In this case, the Beat Type is designated as Unknown, which means that the parameters that are applied in an ischemia test (e.g. ST shift threshold) are generic parameter values that represent a generic Beat Type.

The generic parameter values may be calculated in different ways. For example, the generic parameter values may be a weighted average of the parameter values for upright and supine type beats, a threshold alarm value can be calculated as:

=(average supine final deflection_slope*k1)+(average standing final deflection_slope*k2), where “average supine final deflection slope” is the running average of final deflection slope when the patient is supine, “average standing final deflection slope” is the running average of final deflection slope when the patient is standing, and k1 and k2 are constants related to the amount of time the patient is expected to be supine and standing, respectively.

Alternatively, the generic parameters may be chosen as worst case parameter values to avoid false positives. For example, if an ST shift alarm threshold is +10% for supine and +20% for standing, then the 20% value may also be used for Unknown beats. This worst case choice would decrease the sensitivity of the test (when the patient is actually supine) but help maintain a good specificity.

Additionally, the waveform feature values for the Unknown Beat Type may be adjusted by adding (or subtracting) a constant to align the data so there are no sharp jumps on a trend graph. For example, the size of the shift in waveform feature value attributable to the change in axis may be determined (e.g., the quantity Δ in FIG. 10) and then subtracted from the waveform feature values for the Unknown Beat Type (i.e. shifting down the entire portion of the plot in FIG. 10, which occurs after the ‘jump’ caused by lying down, by the quantity Δ in FIG. 10).

These generic parameter values (e.g. thresholds) may still result in a correct diagnosis of ischemia, since the ischemia is sufficiently severe to change the QRS enough that the system can not classify the beat. Such severe QRS changes are likely to cause an ischemia test (using the generic parameter values) to indicate ischemia. However, if ischemia is not detected, according to one embodiment of the present invention, the diagnostic device 5 may respond to multiple occurrences of an Unknown Beat type by generating a relatively low priority alarm indicating that more information is needed to classify beats. The alarm could instruct the patient to input information regarding the patient's state (e.g. the patient could input his/her posture) and/or employ additional sensors (e.g. by wearing a vest with a high electrode density) to provide additional diagnostic information. Alternatively, if diagnostic device 5 can contingently employ additional sensors (e.g. implanted electrodes) or a more computationally expensive diagnostic scheme than is normally utilized, then the existence of an Unknown Beat Type could trigger utilization of these additional resources. In other words, occurrence of beats classified as Unknown Beat Type can contingently lead the device implement operations which are normally not done either in order to save energy, improve patient comfort, or due to similar considerations.

When a patient is active, there may be considerable variation in waveform feature values due to mechanical and biological sources of noise including, for example, the movement of the heart with respect to the torso. In the case where there is significant variability in QRS waveform feature values (e.g. the standard deviation of the primary, deflection amplitude over 10 beats exceeds a specified threshold), then the Beat Type is considered “Active”. To apply an ischemia test to an Active Beat Type, generic parameter values (discussed above) are employed.

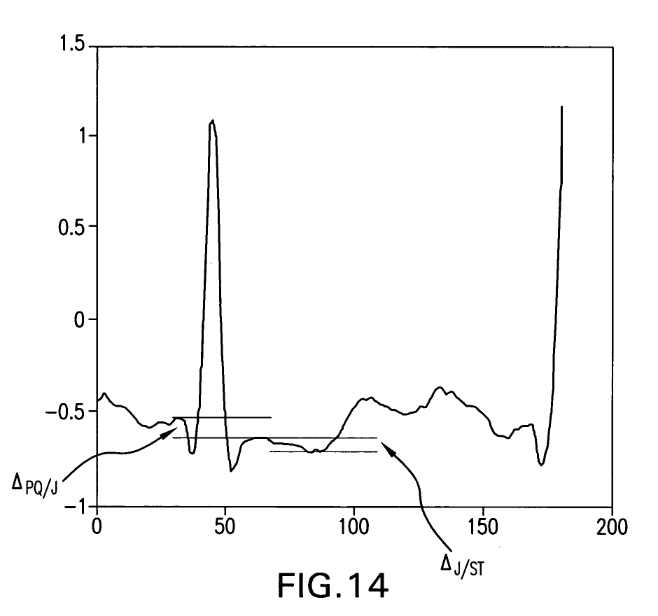

In the preferred embodiment, histograms, trends, and summary statistics such as running averages are kept of certain waveform feature values adjusted for Beat Type and heart rate. For example, a measurement of the difference between the QRS inflection points, which is often at least partially affected by ischemia, is tracked. This difference is identified as the quantity Δ_{PQ/J} in FIG. 14, which shows a sample electrocardiogram waveform. This value may change somewhat according to patient posture. The shift in this value caused by postural changes (and possibly other sources of change) is removed by adding (or subtracting) a constant to align the data so there are no sharp jumps on a trend graph of the ST/TQ difference (Δ_{PQ/J}). This addition (or subtraction) is performed only in the cases when the Beat Type is not Active. For Active Beat Types, the ST/TQ difference will fluctuate for similar reasons that QRS values fluctuate when a patient is active. Taking a running average will tend to mitigate the effects of these fluctuations. For all Beat Types, the ST/TQ difference (Δ_{PQ/J}) is adjusted for heart rate, preferably according to self-norm data.

### Flowchart

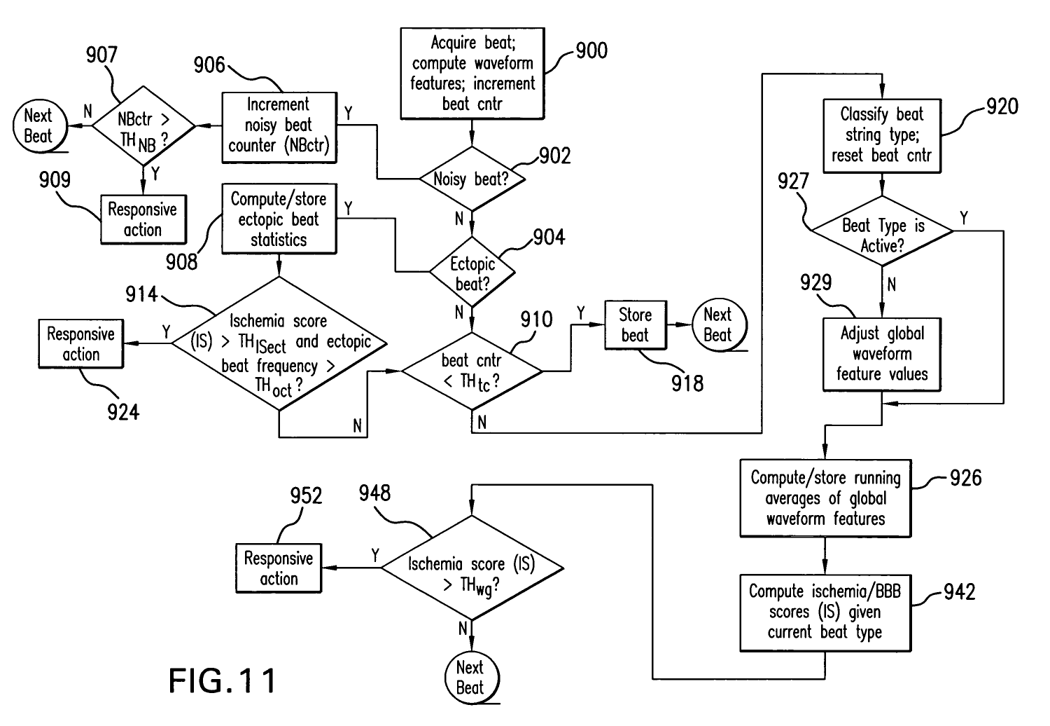

FIG. 11 is a flowchart of the preferred ischemia/bundle branch block detection scheme according to the present invention. The flow chart is implemented by the architecture show in FIGS. 1 and 2. It may also rely upon the logic indicated for evaluating the features of the Table in FIG. 9.

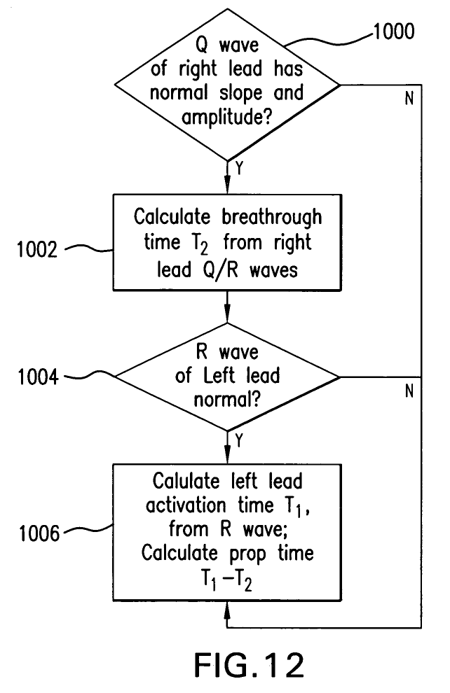

In block 900, the system acquires waveforms corresponding to leads 15 and 12. For convenience, it will be assumed that a single beat is acquired and processed. In practice, it may be desirable to acquire a number of beats and process them as a group, as described in U.S. patent application publication number 20070093720, which also discloses details regarding the acquisition of waveforms. The following waveform features for each waveform are computed: the slopes and amplitudes of the initial, primary and final deflections, ST segment duration and slope, ST segment beginning and end values, T wave amplitude and RR interval between the current and previous beat (which requires that the waveform include the previously analyzed beat). In addition, the propagation time T_{B} is computed, as further described with reference to FIG. 12.

In addition to the waveform features which are described herein, spectral and time-frequency analysis can also be accomplished. Conversions of the data from the time domain into domains such as factor-space using principal component analysis are also known. When more than two leads are used, more complicated methods of spatial analysis and surface mapping can be used to map both time domain data as well as the transforms. It is understood that these other strategies can be used to evaluate essential the same features described here (as well as additional features) without departing from the described invention. For example, using phase information in the frequency domain can be substituted for measuring peaks of the cardiac waveform in the time domain. Further, as is known in the art, both linear and non-linear component analysis can be used to classify QRS complexes (and ST segments).

Many methods are known for extracting the QRS complex from an electrocardiogram signal. According to the present invention, the preferred method involves extracting the inflection points indicative of the beginning and ending of the QRS complex. These points define the Δ_{PQ/J} value shown in FIG. 14. This may be done by imposing a smoothness criteria based on the second derivative of the signal. A time-point is considered as a possible inflection point if: (i) both the first and second derivatives of the point are less than specified thresholds; (ii) neighboring points on one side (e.g. to the left side encompassing prior time-points) are “flat”; and (iii) neighboring points to the other side (e.g. to the right, meaning after in time) are “not flat” and “far away from” the current point, i.e. indicative of being part of the QRS complex. Various Hjorth parameters can also be applied both before and after band-pass filtering, in order to add additional constraints to those points treated as inflection points.

To define “flat”, “not flat” and “far away from”, a function F is constructed that is equal to cumulative sum of the a proxy for waveform second derivative:

$$F = {\sum }_{i} \vert {V}_{i + 1} - 2 {V}_{i} + {V}_{i - 1} \vert ,$$

where Vᵢ is the value of the electrical signal at time ‘i’ The value of F jumps at the onset of the QRS and increases smoothly until the end of the QRS complex. Thus, by examining the difference in F between a current point and its neighbor, it is possible to determine “how far” the neighbor is from the current point. For example, F(j+10)−F(j) is the difference in F between sample j and the 10^{th} sample after j. If the neighbor is “far away”, as defined as being greater than a selected a parameter value, the neighbor may be part of the QRS and the current point may be an inflection point. The second derivative tests can be implemented by taking a running average of the second derivative to smooth this measure somewhat.

The location of the neighbor that is “far away” can be tracked. In other words, if the neighbor that was “far away” is after the current point, then the current point is possibly just before a QRS complex, whereas if the neighbor that was “far away” is before the current point, then the current point is possibly just after a QRS complex. Thus, possible “before QRS” and “after QRS” groups may be defined.

To further enhance the jump in the function F at the onset and offset of the QRS, the second derivative function u=|Vᵢ₊₁−2Vᵢ+Vᵢ₋₁| is essentially filtered to remove or decrease values of u in the range of p and t waves. This filtering will of course remove values of u in the QRS range but the net result is an overall accentuation of QRS u values. A preferred filter is to set u=0 for u<uₜₕ, and then obtain a “filtered”/enhanced signal (u_{f}=u²), where gₜₕ is an empirically derived, signal dependent constant. (For at least some of the signals in the Long Term ST Database available at physionet.org, a uₜₕ value of 0.3 proved effective.)

Another possible filter u_{f} is to take higher order powers of u, e.g., u³. (In the case, u need not be set equal to 0 for u<uₜₕ.). In any event, u_{f} should have the appearance of a stair case, with the corners corresponding to the onset and offset of the QRS complex. These coverns may be found in any number of ways. One way involves taking the derivative u_{f} of the u_{f} function, normalizing its maximum value to 1 (for example), and locating the points in the QRS complex as those points where u_{f} is greater than a certain threshold. The corner points are then the first and last points of all the points in a particular QRS complex.

If g is computed by taking the difference between a first order derivative function q=|Vᵢ−Vᵢ₋₁| (a centered difference function may also be used), then it may be desirable to effectively low pass filter q to remove high frequency noise. (If the signal has been preprocessed with an appropriate low pass filter, then the filtering of q may not be necessary.) One example of a simple low pass filter is a five point moving average of q, resulting in the function q_{f}. The second derivative function u may then be computed as |qᵢ−qᵢ₋₁|.

Once all possible candidates for inflection points are determined, they are grouped according to their proximity to one another. For example, one group of inflection points will be just before a particular QRS complex and another group will be just after the QRS complex. For example, samples 10-20 may form one group just before a QRS complex and samples 80-100 may form another group just after that QRS complex. The inflection point chosen from any particular group is preferably the point with the smallest first or second derivatives out of all the points from the group. The result of the selection process is a set of possible inflection points (e.g. sample 15 from group 1 and sample 83 from group 2 so the set is {15, 83}). Out of this set, consecutive members of the set are chosen as inflection points if they are separated by an appropriate number of samples (e.g. preferably corresponding to a QRS width of between 60-160 ms).

Since the waveform points just after a large T wave may qualify as candidate inflection points than a large P wave, the groups before a QRS complex may be more reliable markers of the proximity to a QRS complex. For example, if a QRST waveform spans samples 1-700, there may be three groups of samples that qualify as possible inflection points: [10-20], [80-100] and [300-320], where the first group is designated as a “before QRS” group and the latter groups ([80-100 and 300-320]) are “after QRS” groups. The following intra-group selection process results in the selection of samples 15, 83 and 312, with sample 15 labeled as “before QRS” and the others “after QRS”. The sample 15 is selected, because it is the only “before QRS” sample, and the first sample after it (sample 83) in the set {15,83,312} is selected as the actual “after QRS” inflection point.

In some instances, more than one candidate inflection point may be identified by the algorithm as an ‘initial’ or other type of inflection point. In this instance the average of the two candidates may be calculated. If no inflection point is found, or the inflection point selected seems to be out of range, the data for that beat may be ignored by the algorithm.

Baseline correction is preferably performed by fitting separate polynomials to the PQ points (i.e. the left side QRS inflection point shown in FIG. 14) and the T wave maxima (less its mean) over four beats, averaging these polynomials, and subtracting them from the waveform to obtain a baseline corrected waveform.

Also in block 900, a beat counter is incremented. The role of the beat counter will be described with reference to block 910.

In block 902, the system determines whether the acquired beat is noisy by analyzing the waveform features of the waveforms corresponding to leads 12 and 15, respectively. If either waveform exhibits non-physiological characteristics (e.g. amplitudes/slopes out of the physiological range), then the beat is classified as a noisy beat. Noisy beats are processed by block 906, which creates a noisy beat summary. A noisy beat summary may be comprised of the proportion of noisy beats within a specified period or across a selected number of beats. If the noisy beat summary value is above a predetermined threshold, as determined in block 907, appropriate responsive action (e.g. sending an alert signal may to the patient) is taken in block 709. Otherwise, the next beat is processed.

If the beat is not classified as noisy, block 904 determines whether the beat is ectopic based on, for instance: (i) RR interval variability; (ii) features of the QRS complex, in particular, the duration of the QRS complex and the ratios of the initial, primary and final deflections; and (iii) T wave polarity. If the beat is ectopic, control passes to block 908, computes a ectopic beat summary. Control then passes to block 914, which checks whether the current ischemia score (a value between 0 and 1, as will be further described below), is above a selected threshold (TH_{1S,ect}) and the ectopic beat summary is above a certain threshold (TH_{1S,ect}). This check is performed because ischemia can cause ectopic beats; thus, the existence of ectopic activity may be used to tip the balance toward a determination of ischemia when the ischemia score (IS) is equivocal. If the IS and ectopic beat frequency both exceed their respective thresholds, control is transferred to block 924, where an appropriate responsive action (e.g. alerting the patient and/or medical practitioners through telemetry as further described in U.S. patent application publication number 2004/0215092) is generated. Otherwise, control is passed to block 910.

Block 910 determines whether a beat counter (beat cntr) is greater than a threshold (TH_{bc}) that determines how many beats will be analyzed a group/string. A possible value for TH_{bc} is 10. If the beat counter is less than TH_{bc}, control passes to block 918, which stores the beat. The next beat is acquired. If the beat counter is equal to TH_{bc}, control passes to block 920, which resets the beat string counter and classifies the Beat Type of the string of TH_{bc} (e.g. 10) beats.

Control passes to block 920, which classifies the Beat Type of a string of beats that includes the current beat. As previously mentioned, different Beat Types correspond to different patient states, each with an associated set of expected waveform feature values. To determine the Beat Type of the current string of beats, the beats' waveform features, preferably QRS slopes and amplitudes, are compared to the expected values for different Beat Types. Again, it is emphasized that this comparison is based on matches between both the waveform recorded by lead 15 and the waveform recorded by lead 12 and corresponding expected values. Information (e.g. posture information) provided by other sensors (e.g. a level detector) may be used in conjunction with (or as an alternative to) the waveform information to classify the beat.

The first step in classifying the string is to determine the variance of the waveform features (e.g. primary deflection amplitude and initial/primary deflection amplitude ratio) being analyzed in the current string of beats. If the variance exceeds a specified threshold (which may be set only for duration in relation to being above a select variability level, only for level of the variability, or for a combination of the two), then the Beat Type for the string is defined as Active, based on the assumption that the variance of the data is a result of the patient being active, which, as discussed above, increases the variability of the data (by, for example, causing movement of the heart with respect to the recording leads). Otherwise, there is a steady string of beats whose Beat Type may be characterized as one of the known Beat Types (e.g. supine) or possibly an Unknown Beat Type.

Within a beat string, there may be a transition between two different Beat Types. The two different beat types may cause the variability of analyzed QRS features to exceed threshold so that the string is characterized as “Active” even if the patient is not really active. However, this “mischaracterization” of a single string of beats does not cause any problems, and can be constrained by the algorithm to decrease its occurrence.

Control then passes to block 927, which checks whether the beat string has been classified as Active. If so, control passes to block 926. If the beat string was not classified as Active, meaning that the present string of beats is steady, then control passes to block 929, which adjusts various waveform feature values according to the Beat Type. For example, the TQ/ST difference (Δ_{PQ/J} in FIG. 14) is preferably adjusted. Assuming that the current Beat Type is supine, and assuming that the supine state is known to cause a shift of −0.1 mV in the TQ/ST difference, then 0.1 mV is added to the TQ/ST difference to compensate for the shift that is solely attributable to posture.

Instead of making a pre-specified adjustment, to allow the system to adapt to slow changes and/or to handle Unknown Beat Types, the shift in waveform feature values may be computed on the fly by measuring the amount of the shift and subtracting this amount (e.g. the amount Δ in FIG. 10). If this method of adjusting for shifts is adapted, the system must be able to measure the shift (Δ in FIG. 10). Since the shift will most likely occur within a given string of beats, the system is configured to detect when the transition between Beat Types occurred within a given string of beats. For example, if strings are defined to consist of 10 beats, the shift may occur between beats 6 and 7 in a current string of beats. The subsequent string of beats will be of the same type as beats 7-10 in the current string. To determine the size of the shift, the system must store the current string so that it can be concatenated with the subsequent string. The resulting “concatenated-string” will consist of 20 beats, and the system can then determine all of the beats (beats 7-20) that are of the same Beat Type (e.g. supine). The system can then compute the amount of the shift in waveform feature value that associated with the shift to the new Beat Type (e.g. supine), and can compensate for this shift.

Control passes to block 926, which updates the running averages of waveform feature values (which may be adjusted in block 929) listed in the Table in FIG. 9. Running averages may be computed by averaging beats, and determining the waveform value for the averaged beat, as described by Pueyo et al. (2005), which also discloses exponential averaging, with weights updated according to the noisiness of the data. Alternatively, waveform feature values may be computed for each beat, and the running average (standard or exponential) of these values may then be computed. The number of beats over which to average a waveform feature value may be a function of the noisiness of the data.

In addition to computing running averages over a relatively small number of beats (e.g. 10 beats) for noise reduction purposes, running averages may also be taken over a relatively large number of beats so as to provide an easily computable index of relatively long term trends for the data. For example, a 180 beat running average of the TQ/ST offset may be computed. This running average may be used in an ischemia test, as described below. Preferably, the value of this running average is stored periodically, for example every 5 minutes, to allow a determination of how rapidly this running average is changing. Alternatively or in addition to such periodic storing, running averages may be taken of the slope of the running average curve, thereby providing a measure of how rapidly the underlying feature (e.g. TQ/ST offset) has changed. Again, this running average may be used in an ischemia test. For example, if the running average of the TQ/ST offset reaches a value indicative of a physiological problem, then running average of the slope of the TQ/ST running average curve may be checked to determine if the TQ/ST offset has changed rapidly, likely indicative of ischemia, or has changed gradually, indicative of other problems such as pericarditis, hyperkalemia etc.

Running averages may be computed using averaged beats, or by averaging the measures of sets of single beats, although the prior embodiment is preferred in the case of measurement of ST-segments. For running averages that are taken over a long time/large number of beats, exponential averaging may be used, which avoids the requirement of storing a large number of samples.

In addition to implementations using running averages, other statistical features and measures (trending, estimates of variance such as guardbands, estimates of distribution, density functions, and skewness) may be used additionally, or alternatively, to detect ischemic events.

Control passes to block 942, which computes two ischemia scores, one for subendocardial ischemia and one for transmural ischemia. Although a number of features may be combined to produce this score, a general formula is preferably applied is as follows:

$$IS = max \{ {f}_{j} \} , where {f}_{j} = {\sum }_{i} {w}_{i} * {g}_{i} ( {x}_{i} , {μ}_{i} , {t}_{i} )$$

The ischemia score is formed by taking the maximum value of a set of summation functions (ischemia tests) fⱼε[0,1], where each summation function fⱼ is a weighted sum of functions gᵢε[0,1] that depend on a waveform feature value xᵢ, the expected value μᵢ of the waveform feature value xᵢ, and the threshold value tᵢ at which the waveform feature value xᵢ−μᵢ is considered to be definitely (or highly likely) in the ischemic range. (Stated differently, each ischemia test fᵢ may be considered to apply weights wᵢ to different functions gᵢ. The functions gᵢ pertain to waveform feature values, so each ischemia test fⱼ may be considered to apply weights wᵢ to waveform feature values xᵢ.)

In addition to being a particular stored value, “tᵢ’ can also be determined as, for example, a self norm, a population norm, adjusted as a function of the variance or standard deviation of the measure, which may be a recently computed estimate for a prior period, can be adjusted as a function of patient state, as a function of axis shift. The weights wᵢ are chosen so that the sum (fⱼ) never exceeds 1; as a simple example, if there are N parameters in a sum (i.e., i varies from 1 to N), then wᵢ may be chosen as a constant (1/N). The parameters μᵢ and tᵢ will preferably depend on the current Beat Type.

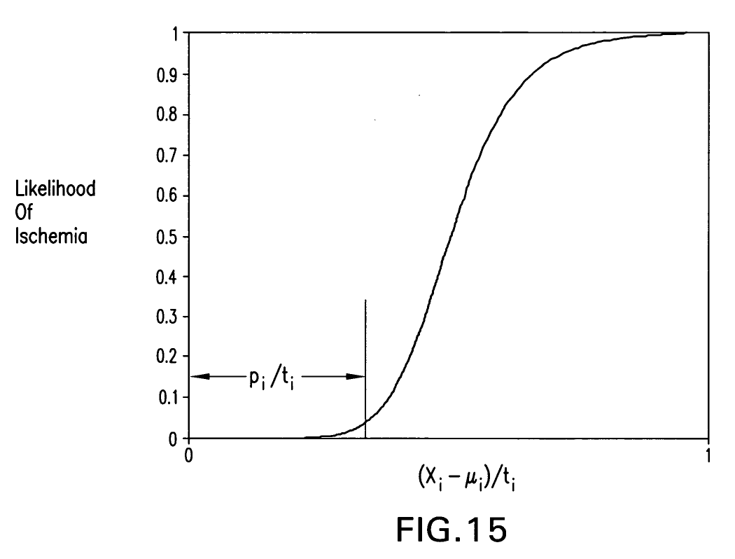

Each function gᵢ is chosen to be small when the waveform value xᵢ is equal to its expected value μᵢ and converge towards 1, or equal 1, when waveform value xᵢ is equal to its threshold value tᵢ. For ease of discussion, it will be assumed that tᵢ>μᵢ, and that gᵢ is chosen to approximate 0 when xᵢ<μᵢ. In other words, any increase in the value xᵢ is considered to correspond to a greater likelihood of ischemia. In reality, since decreases in a waveform value, or any change (positive or negative) in a waveform value, may correspond to a greater likelihood of ischemia, rather than measuring ‘x’ the equations may measure transforms such as ‘1/x’, ‘constant-x’, or the like, including non-linear transformations as is well known in the art. One easy solution is to implement a choice for gᵢ is min[(max(xᵢ−μᵢ,0)/tᵢ),1]. Another possibility is a sigmoidal type function (xⁿ/(1+xⁿ)), as shown in FIG. 15.

As described in pending U.S. patent application Ser. No. ______, the form of the IS described above allows for a flexible “OR” type ischemia test. For example, one fⱼ may require moderate deviations from normal or expected values in both QRS and ST features, while another fⱼ may require more significant changes in only ST features when the QRS features are assessed as normal while still another fⱼ may be based only on QRS features

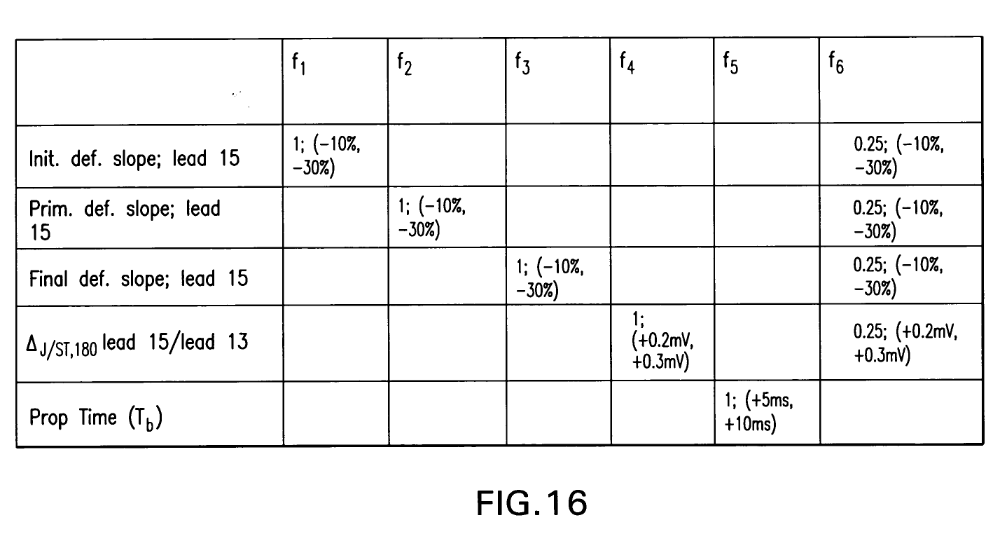

FIG. 16 shows an example of the components of an ischemia score (IS) for the LAD. There are five functions fⱼ, each of which is associated with a separate column in the table shown. The weights wᵢ for each fⱼ, and the parameters for each gᵢ, which is assumed to be a function of the type shown in FIG. 15, are arranged as rows. For each table entry, the weights are shown to the left of a semicolon and the parameters for each gᵢ, are shown in parenthesis. For example, the function f₁ is based solely on initial deflection slope in lead 15. Unless otherwise indicated in this example, waveform feature values such as initial deflection slope are considered to be 30 beat running averages. The weight for initial deflection slope is thus 1 since there are no other waveform values associated with f₁. The number in the left side of the parenthesis indicates that a decrease of 15% from the normal value of the initial deflection slope is considered to be on the cusp of abnormality. Thus, if the slope decreases more than 15%, the value of f₁ should start to increase appreciably.

The increase in f₁ is implemented by the sigmoidal function shown in FIG. 15. In that figure, the function increases rapidly when the difference in waveform feature value xᵢ from its normal value μᵢ, exceeds pᵢ. In the current example regarding initial deflection slope, pᵢ is ‘−15%’ (15% less than) of the normal value and the threshold tᵢ value is −30% (30% less than) the normal value. −30% is the second value in the parenthesis in the table shown in FIG. 16. Here, only decreases in slope are considered abnormal. Increases in slope from the expected value can all be mapped to a likelihood of ischemia of 0.

In FIG. 15, a particular sigmoidal function is shown in which pᵢ appears to be a fixed fraction of tᵢ equal to 10%/30%=⅓. However, for other values of pᵢ and tᵢ, different sigmoidal functions must be selected. The quantity pᵢ/tᵢ, should be interpreted as the point where a waveform parameter has reached the border of what is considered a normal value.

Returning to FIG. 16, the entries for primary deflection slope (f₂) and final deflection slope (f₃) are similar to the entry just described for initial deflection slope. The entry for f₄ involves the quantity Δ_{PQ/J,180} which means that the value Δ_{PQ/J} (see FIG. 14) is averaged over 180 beats. For this waveform feature value, the (pᵢ,tᵢ) table entry is defined in terms of absolute values (mV), not percentages (as was the case for the initial, primary and final deflection slopes in f₁-f₃) Similarly, the (pᵢ,tᵢ) pair for propagation time (f₅) is given in terms of absolute time (ms). “pᵢ,tᵢ” pairs could also be based up statistically derived thresholds, such as the standard deviation (σ) of a waveform feature value, in which case a reasonable (pᵢ,tᵢ) pair would be (2σ, 3σ).

The last function (f₆) in the table in FIG. 16 is based on the values of four waveform features, each of which is assigned equal weighting. The final ischemia score (IS) would be derived based upon an evaluation of the six functions and can utilize the maximum score across the set of values of the derived by the functions f₁-f₆.

The table shown in FIG. 16 is one example of an IS that may be based on the waveform features shown in FIG. 9. Many other types of IS′ can be constructed from those waveform features or even complex relationships between those features. Particularly, an IS may also be based on temporal relationships between waveform feature values. It is known (Pueyo et al., 2005) that repolarization parameters (ST/T) change rapidly after a total occlusion while changes in QRS parameters occur somewhat later, approximately 2 minutes after occlusion. The relative timing of these changes (shifts) can be taken into account with a particular fⱼ. In particular, if (some portion of) the QRS slope has shifted by more than a predetermined threshold within a specified lag after the ST level had shifted by more than a predetermined amount, then the ischemia test will indicate a positive result.

The tracking of the relative timing of ST and QRS slope shifts may be done in a number of ways. For example, the QRS slope shift at any particular time may be defined as the current QRS slope minus the QRS slope at a first prior time (e.g. two minutes ago). The ST shift at any point in time may be defined as the current ST level−the ST level at a second prior time (e.g. 1 minute ago). The QRS slope shift may be combined in an ischemia test with the maximum ST shift that occurred between a third prior time (e.g. 1-3 minutes) prior to the current time. The maximum ST shift which occurred within this third time window may be determined by maintaining the ST shift as a running variable which can be corrected for axis shifts.

The change in a waveform feature value over a long period of time may be tracked and result in an appropriate alarm as described in U.S. patent publication number 20050113705 to Fischell et al. Such long term changes could be indicative of worsening chronic ischemia or other conditions such as pericarditis.

In the case where the current Beat Type is Unknown, the threshold values tᵢ are chosen to represent the largest ischemic values that may be expected regardless of the patient's state (e.g. sitting, lying down etc.) For example, regardless of patient state, which in this case includes axis shifts, the T wave recorded by either lead 15 or 12 should not be flat or inverted. As a further example, the absolute value of the primary deflection slope should not be less than three standard deviations below the lowest normal absolute value of the primary deflection slope across all patient positions (e.g., sitting, standing, laying on the left or right side, laying or on stomach or on back).

Either left or right bundle branch blocks (BBB) may cause significant changes in QRS slopes, the shape of the QRS complex and/or the ST/T segment. A rapid onset BBB condition, as indicated by the short term shifts in the values of these features, may be indicative of severe ischemia. Thus, detection of rapid onset BBB events is not regarded as a false positive with respect to a number of conditions which may be measured and including the case of ischemia.

The IS for subendocardial ischemia concerning the most recent 5 or 10 minutes (or more) of data is preferably stored. The stored subendocardial ischemia IS may form part of the IS for transmural ischemia. As is known, transmural ischemia often evolves from subendocardial ischemia. The transmural IS may be first computed without reference to subendocardial ischemia. Then, a new, weighted transmural IS may be formed as a weighted average of the previously computed transmural IS and the maximum subendocardial IS over the last 5 or 10 minutes of data. The final transmural IS is then chosen to be the greater of the initially computed transmural IS or the transmural IS weighted by the subendocardial IS. Alternatively, or in addition to weighting the previous subendocardial IS, the transition of a particular waveform feature value between subendocardial and transmural ischemia may also be taken into account for the transmural IS. In other words, the recent historical record of a particular waveform feature can be evaluated for size, rate, pattern, temporal pattern, and rate of change, with respect to transitions of at least one waveform feature. In this case, the decrease from baseline of the T wave amplitude may be indicative of subendocardial ischemia and a subsequent increase beyond baseline, within a selected timeframe, may be indicative of a transition to transmural ischemia associated with a particular artery. When more than one lead is available, the transitions can be evaluated within or across leads.

One of the waveform values that preferably factors into the transmural IS is propagation time between lead 12 and lead 15. FIG. 12 is a flowchart of a possible method for calculating this propagation time. Block 1000 checks whether the initial deflection of the right lead (503) has a normal slope (e.g. the slope and amplitude are within 2 standard deviations of the mean slope and amplitude, as determined by an initial data acquisition phase, across some or all tested patient states/positions). If so, in block 1002, the peak of the initial deflection is determined and the associated time of the peak is defined as the epicardial breakthrough time T_{b}. Different fiducial times, such as the maximum negative slope of the R wave 902 (FIG. 3b), could be chosen. In block 1004, the system checks the R wave amplitude and slope associated with the left lead 15. The R wave check is preferably similar to the final deflection check performed in block 1000. If the R wave passes the normalcy test, in block 1006, the maximum negative slope of the R wave is estimated, and the associated time of this slope is defined as the left lead activation time T₁. Again, different fiducial time markers could be chosen, such as the nadir of the R wave of the left lead 15. Propagation time is then defined as Tₗ−T_{b}.

### Auto-Calibration; Dimensionless Embodiment

Because the absolute values of various waveform features are analyzed in one embodiment of the present invention, it is desirable to make the measurement of the absolute values as accurate as possible. The measurement of the absolute values could be affected by impedance changes at the electrode site or other device related factors. To test whether a particular lead has been adversely affected by these factors, a test current pulse may be periodically applied across the lead and the resulting measured voltage drop measured and compared to a reference value. This test pulse can be set to occur at a particular phase of the cardiac cycle and when a patient is in a particular state (e.g. supine), in order to cause the transmission to isolate the effects of device effects, as opposed to patient state effects, on measured voltages. To lessen the electrical noise associated with the heart and other body parts, the pulse is preferably sent during a particular phase (e.g., the TP phase) of the heart cycle, while the patient is supine and preferably asleep. Minor changes from the reference value could be incorporated into the detection algorithms by scaling all appropriate absolute voltage quantities. Further, a major change which is beyond a selected normal range would cause an alert to be sent to the patient which indicated that the patient should have the device checked by a qualified person.

As an alternative to periodically calibrating the device, waveform features may be normalized as follows:
1. Initial, primary and final deflection slopes: instead of calculating the slopes, the times between the pertinent inflection points may be computed.
2. All amplitudes could be normalized by primary deflection amplitude, as disclosed (for ST amplitudes) in U.S. Pat. No. 6,609,023 to Fischell et al., which is incorporated by reference herein. In this case, as in the '023 patent, the primary deflection factor used for normalization should be a baseline, normal (non-ischemic) primary deflection that is updated periodically, If the primary deflection amplitude (for a given beat type) has been changing over the order of minutes as determined by the moving average primary deflection tracking described above, then ischemia may be detected and the primary deflection is not suitable as a reference.
   Chronic and/or Non-Transmural Ischemia

The electrode positions and ischemia detection scheme discussed above are preferably optimized for detecting an acute transmural ischemic event. Subendocardial ischemia, whether chronic or acute, likely causes less drastic QRS and propagation time changes than acute, transmural ischemia. Subendocardial ischemia (at least the chronic type) is known to cause different ST shift patterns than acute, transmural ischemia. (Hopenfeld B. ST segment depression: the possible role of global repolarization dynamics. Biomed Eng Online. 2007 Feb. 9; 6:6.)

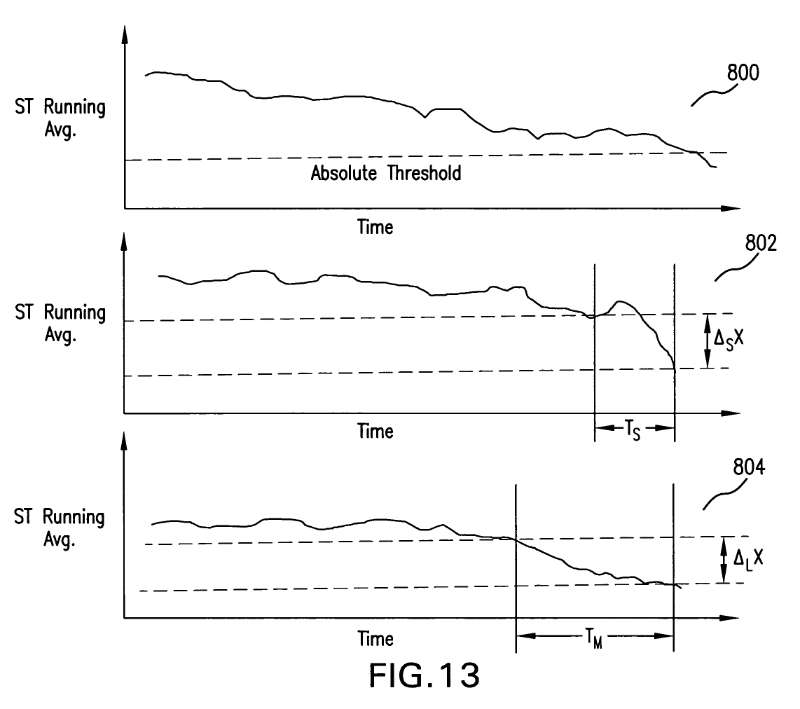

Early ST segment (shifts caused by subendocardial ischemia may not be significant in waveforms recorded by lead 15. Lead 12 may record somewhat of a trend toward negative ST segment values (but not negative absolute ST segment potentials.) (Early ST segment means approximately 60-80 ms after the J-point. The J-point is essentially the end of the final deflection.) A long term trend of ST segment potential as a function of heart rate, as described in U.S. patent publication number 20050113705 to Fischell et al., can be implemented to detect this type of chronic ischemia. This type of long term tracking is shown in plot 800 in FIG. 13. Short and medium term ST segment changes may also be tracked, as shown in plots 802 and 804 respectively. A significant short term (and rapid onset) shift, even in the absence of QRS changes, could signify an acute subendocardial ischemic event, especially if the shift occurs at a relatively low heart rate. (The plots 800, 802 and 804 are all assumed to track the ST trend for a particular heart rate range.)

Lead 15 may register changes in initial, primary and final deflection amplitudes in the presence of subendocardial ischemia. Further, in a healthy person, an increase in heart rate may be associated with changes in initial, primary and final deflection amplitudes registered by lead 15, as suggested by the data of Miller et al. (Circulation; 632-645, 1980). Specifically, an increase in heart rate may increase initial and final deflection amplitudes and decrease the primary deflection amplitude. If a patient has subendocardial ischemia, this increase may not occur (or occur not as much as normal), so the lack of normal changes in these amplitudes with increases in heart rate is a marker of subendocardial ischemia. (This marker for ischemia is part of the Athens score as applied to the standard 12 lead ECG. Michaelides et al., Am Heart J. 1990 August; 120(2):292-302.)

According to the present invention patient's baseline body surface map may be taken at normal and high heart rates to determine the torso locations of the greatest initial, primary or final deflection changes. Based on this information, electrode positions may be adjusted to increase sensitivity to changes in initial, primary or final deflection amplitude. Subsequently, if the system determines that these baseline changes in initial, primary or final deflection amplitude are not occurring with increases in heart rate, the system may determine that the patient is experiencing subendocardial ischemia.

### Distributed Wireless Sensors/Use of Activation Mapping by Bipolar Electrodes

In an alternative embodiment, a method of detecting cardiac abnormalities can utilize a plurality of electrodes which are distributed across the torso surface. A plurality of small implantable devices (SIDs) are likewise distributed throughout the torso; and each SID is coupled to at least one nearby pair of electrodes. The pairs may be either separate from one another or daisy chained. Each of the SIDs comprises an amplifier, filter, analog-to-digital converter, a digital processor, an antenna, and communications circuitry.

Using this distributed array, deviations from a normal activation sequence may reveal various cardiac pathologies. In turn, information regarding the normal activation sequence may be gleaned from measurements taken from the torso surface. One type of measurement from the torso surface involves detection of local (torso) activation time, which may be defined with some appropriate metric such as the time when the maximum slope occurs in an electrogram during the initial deflection that represents the passing of the cardiac wave across an electrode (unipolar) or between electrodes (bipolar).

### Track ST Shift Progression Between Leads

As transmural ischemia expands, ST shifts as recorded between selected of bipolar leads may decrease, while ST shifts between other spaced electrodes may increase. For example, the ST-shift for leads having more closely spaced electrodes (Lc) may decrease while those with electrodes spaced further apart (Lf) may increase. By comparing the ST shift between Lf and Lc combinations, the detection and even quantification of increasing ST shift can be obtained. In the current embodiment a montage comprised by pairing the input signals of electrodes 13 and 14, and 16 and 17 can be compared to that of 13 and 16 and 14 and 17, and if the difference between the ST shifts measured between the two montages changes over time, then this can indicate the expansion of ischemia.

Combination Intracardiac/Extracardiac Embodiment

The data obtained from the dual lead electrode configuration described above can be combined with electrograms obtained from an intracardiac lead. The two types of data can be used in complementary fashions in order to provide a clearer indication of coronary distress than either dataset alone. In addition to detection and diagnosis of cardiac abnormalities, this combination of data can be used to detect axis shifts. Since an intracardiac lead placed in the ventricle (referenced to the ‘can’) will normally not show any signs of axis shifts, when amplitudes vary at particular leads but not within the intracardiac data then these differential amplitudes can be used to detect axis shifts. These detected shifts can then be compensated for in the analysis of the data. Similar to the extracardiac configuration, this configuration can be calibrated for each patient while they are in different postures, or during/after they transition into these postures, in order to provide template values for subsequent categorization of posture during operation of the device.

Derivation of Additional Electrode Configurations Electrodes 16 and 17 of lead 15 and electrodes 13 and 14 of lead 12 are normally configured for intra-lead bipolar sensing. However, using analog means in the device (or in some cases a mathematical equation) the potentials sensed at an electrode within one lead (e.g. electrode 17) can also be referenced to (subtracted from) potentials sensed at an electrode (e.g. electrode 16) within the other lead. Also, any of the electrodes 13, 14, 16 and 17 can be referenced to the ‘can’ or to an intra-cardiac lead, if provided. The use of re-montaging can be used to detect local field potentials more robustly with a limited set of leads and can be used to generate virtual electrodes (e.g. using spline interpolation techniques). The data of the Table in FIG. 9 can be extended to these additional sets of bipolar configurations and virtual electrodes.

More than two leads may be used in accordance with the teachings of the present invention. Additional leads could be used for many purposes, including enhancement of propagation time data. For example, the local activation time for an additional lead could be defined similar to the manner in which activation time was defined for lead 12. The propagation time between lead 12 and lead 15 could be compared with the propagation time between this additional lead and lead 15. Also, an additional lead could be placed so that the waveforms it records are very sensitive to axis shifts, so that the waveforms from this lead could be analyzed to detect axis shifts.

Various other modifications, adaptations, and alternative designs are of course possible in light of the above teachings. Therefore, it should be understood at this time that, within the scope of the appended claims, the invention can be practiced otherwise than as specifically described herein.

What is claimed is:

1. A device for assessing the condition of a mammalian heart, comprising:
(a) first and second leads adapted to be disposed outside of the heart;
(b) a processor coupled to the first and second leads, the processor configured to:
(i) obtain first and second signals from the first and second leads, and compute first and second waveforms corresponding to the first and second signals, each of the first and second waveforms being characterized by a QRS complex associated with cardiac activation;
(ii) compare features of the QRS complexes of the first and second waveforms to derive a propagation time estimate that is indicative of the speed of cardiac activation through at least a portion of the heart;
(iii) apply a test to detect a pathological heart condition, wherein the test is based on the propagation time; and
(c) a signaling device in communication with said processor for sending a signal to an indicator device when said pathological heart condition is detected.

2. The device of claim 1 wherein the device is configured to compare features of the QRS complexes of the first and second waveforms by computing first and second fiducial time points corresponding to the QRS complexes of the first and second waveforms respectively, and wherein the propagation time estimate is based on the difference between the first and second fiducial time points.

3. The device of claim 1 wherein the test which is based on the propagation time is also based upon the shape of the QRS complex.

4. The device of claim 2 further comprising a third lead for computing a third waveform having a QRS complex by which a third fiducial timepoint can be calculated in order to define a second propagation time and wherein the test is based on a comparison of the first and second propagation times

5. The device of claim 1 further comprising an implantable metal case, and wherein the processor is disposed within the implantable metal case.

6. The device of claim 1 where the signaling device includes a telemetry mechanism.

7. The devise of claim 6 where the telemetry mechanism includes a wireless transmitter.

8. The device of claim 6 where the telemetry mechanism includes a cell phone.

9. The device of claim 1 where the signaling device includes an alarm mechanism.

10. The device of claim 1 where the signaling device is external to the patient.

11. A device for assessing the condition of a human heart, the human characterized by a torso, the device comprising:
(a) a first lead adapted to be disposed outside of the heart in such a manner as to detect the potential difference between anterior and posterior portions of the torso;
(b) a processor coupled to the first lead, the processor configured to:
(i) obtain a first signal from the first lead, and compute a first waveform corresponding to the first signal, the first waveform being characterized by a QRS complex associated with cardiac activation, the QRS complex characterized by an initial deflection, a primary deflection and a final deflection;
(ii) compute a first value that is indicative of the slope of the final deflection;
(iii) apply a test to detect ischemia in a posterior portion of the heart, wherein the test is based on the first value; and
(c) a signaling device in communication with said processor for sending a signal to an indicator device when said first value is external as predetermined threshold range.

12. The device of claim 11 where the signaling device includes a telemetry mechanism.

13. The device of claim 12 where the telemetry mechanism includes a wireless transmitter.

14. The device of claim 12 where the telemetry mechanism includes a cell phone.

15. The device of claim 11 where the signaling device includes an alarm mechanism.

16. The device of claim 11 where the signaling device is external to the patient.

17. A device that is configured to assess the condition of a human heart, the human characterized by a torso, the device comprising:
(a) a first lead adapted to be disposed to measure the potential difference between a first anterior region on the left precordium of the torso and a first inferior region to the left of the first anterior region;
(b) a second lead adapted to be disposed to measure the potential difference between a second anterior region to the right of the first anterior region and a second inferior region to the right of the second anterior region;
(c) a processor configured to obtain first and second signals from the first and second leads, respectively, and to compute first and second waveforms corresponding to the first and second signals, respectively, and to apply a first plurality of tests and a second plurality of tests to detect ischemia, wherein the first and second pluralities of tests are based on values of features of the first and second waveforms, respectively; and
(d) a signaling device in communication with said processor for sending a signal to an indicator device when said features of said first and second waveforms are external a predetermined threshold range.

18. The device of claim 17 where the signaling device includes a telemetry mechanism.

19. The device of claim 18 where the telemetry mechanism includes a wireless transmitter.

20. The device of claim 18 where the telemetry mechanism includes a cell phone.

21. The device of claim 17 where the signaling device includes an alarm mechanism.

22. The device of claim 17 where the signaling device is external to the patient.

23. The device of claim 17 wherein first and second tests that are part of the first plurality of tests involve the application of weights to waveform feature values.

24. The device of claim 23 wherein the first and second tests apply different weights to the same waveform feature value.

25. The device of claim 24 wherein the first test involves a single waveform feature value and the second test involves more than one waveform feature value.

26. The device of claim 25 wherein the first test involves an ST segment related waveform feature value, and the second test involves the ST segmented related waveform feature value and a QRS related waveform feature value.

27. The device of claim 17 wherein first and second tests that are part of the first plurality of tests involve the application of test parameter values to waveform feature values.

28. The device of claim 27 wherein each of at least two of the test parameter values is a threshold value associated with a corresponding waveform feature value.

29. The device of claim 28 wherein first and second tests that are part of the first plurality of tests involve the application of different threshold values to the same waveform feature value.

30. The device of claim 29 wherein the waveform feature value is related to the ST segment, and wherein the different threshold values correspond to opposite polarities of the same waveform feature value.

31. A device for assessing the condition of a human heart, the human characterized by a torso, the device comprising:
(a) a first lead adapted to be disposed outside of the heart in such a manner as to detect the potential difference between a superior anterior portion of the torso and an inferior portion of the torso to the right of the superior anterior portion;
(b) a processor coupled to the first lead, the processor configured to:
(i) obtain a first signal from the first lead, and compute a first waveform corresponding to the first signal, the first waveform being characterized by a QRS complex associated with cardiac activation, the QRS complex characterized by an initial deflection, a primary deflection and a final deflection;
(ii) compute a first value that is indicative of a feature of the initial deflection;
(iii) apply a test to detect ischemia in the right ventricle, wherein the test is based at least in part on the first value; and
(c) a signaling device in communication with said processor for receiving said first value and sending a signal to an indicator device when said first value is external a predetermined threshold range.

32. The device of claim 31 where the signaling device includes a telemetry mechanism.

33. The device of claim 32 where the telemetry mechanism includes a wireless transmitter.

34. The device of claim 32 where the telemetry mechanism includes a cell phone.

35. The device of claim 31 where the signaling device includes an alarm mechanism.

36. The device of claim 31 where the signaling device is external to the patient.

37. The device of claim 31 wherein the feature of the initial deflection is a measure of slope.

38. A device for assessing the condition of a human heart, the human characterized by a torso, the device comprising:
(a) a first lead adapted to be disposed outside of the heart;
(b) a processor coupled to the first lead, the processor configured to:
(i) obtain a first signal from the first lead, and compute a first waveform corresponding to the first signal, the first waveform being characterized by a QRS complex associated with cardiac activation and an ST segment, the QRS complex characterized by an initial deflection, a primary deflection and a final deflection;
(ii) compute a first value that is indicative of the amplitude of the final deflection;
(iii) compute a second value that is indicative of the magnitude and polarity of at least a portion of the ST segment;
(iii) apply a test to detect ischemia, wherein the test associates a greater likelihood of ischemia with changes in both the first value and the second value; and
(c) a signaling device in communication with said processor for receiving said first and second values and sending a signal to an indicator device when said first or second value is external a predetermined threshold range.

39. The device of claim 38 where the signaling device includes a telemetry mechanism.

40. The design of claim 39 where the telemetry mechanism includes a wireless transmitter.

41. The device of claim 39 where the telemetry mechanism includes a cell phone.

42. The device of claim 38 where the signaling device includes an alarm mechanism.

43. The device of claim 38 where the signaling device is external to the patient.

44. The device of claim 38 wherein the changes in both the first value and the second value are in relation to a self norm.

45. The device of claim 38 wherein the changes in both the first value and the second value are in relation to a self norm from within the prior 2 hour period.

46. The device of claim 38 wherein the changes in both the first value and the second value occur approximately concurrently.

47. The device of claim 38 wherein the changes in both the first value and the second value occur for a specified number of beats.

48. The device of claim 38 wherein the test detects ischemia based upon both the amount of the changes in both the first value and the second value and the duration of such changes, such that increases in the amount of changes will result in a positive detection of ischemia over a decreased duration.

49. The device of claim 48 wherein duration is defined by a specified number of beats.

50. The device of claim 48 wherein the test is based on moving averages of the first and second values.

51. The device of claim 50 wherein the moving averages are exponential moving averages.
